(19) United States
(12) Patent Application Publication
(10) Pub. No.: US 2021/0332396 A1
(43) Pub. Date: Oct. 28, 2021

(54) GENETIC ELEMENTS IN ENTEROCOCCUS SPP. TO PRODUCE DOPAMINE

(71) Applicant: Iowa State University Research Foundation, Inc., Ames, IA (US)

(72) Inventors: Mark Lyte, Ames, IA (US); Daniel Nicholas Villageliu, Ames, IA (US)

(21) Appl. No.: 17/250,878

(22) PCT Filed: Sep. 23, 2019
(86) PCT No.: PCT/US19/52440

§ 371 Date: Mar. 18, 2021

Related U.S. Application Data
(60) Provisional application No. 62/734,323, filed on Sep. 21, 2018.

(51) Int. Cl. C12P 13/00; C12N 9/88

(52) U.S. Cl. CPC C12P 13/001; C12Y 401/01025; C12N 9/88

(57) ABSTRACT

The present invention relates to nucleic acid molecules from regions of Enterococcus spp. genomes which are associated with the production of dopamine The invention also relates to proteins encoded by such nucleic acid molecules as well as nucleic acid markers which are associated with high dopamine production. Moreover, the invention relates to uses of such molecules, including, but not limited to, transforming or transfecting cells or organisms with constructs containing the nucleic acid molecules to create cells or organisms with enhanced dopamine production. The present invention is also directed to kits for identifying bacteria which may be capable of producing dopamine based on the detection of the nucleic acid molecules.

### CROSS REFERENCE

The present application claims priority to the earlier filed U.S. Provisional Application having Ser. No. 62/734,323, filed Sep. 21, 2018, and hereby incorporates subject matter of the provisional application in its entirety.

### GRANT REFERENCE

This invention was made with government support under Grant No. N00014-15-1-2706 awarded by the U.S. Department of Defense, Office of Naval Research. The Government has certain rights in this invention.

### FIELD OF THE INVENTION

The present invention relates to nucleic acid molecules from regions of Enterococcus spp. genomes which are associated with the production of dopamine. The invention also relates to proteins encoded by such nucleic acid molecules as well as nucleic acid markers which are associated with high dopamine production. Moreover, the invention relates to uses of such molecules, including, but not limited to, transforming or transfecting cells or organisms with constructs containing the nucleic acid molecules to create cells or organisms with enhanced dopamine production. The present invention is also directed to kits and systems for identifying bacteria which may be capable of producing dopamine based on the detection of nucleic acids molecules.

### BACKGROUND OF THE INVENTION

Dopamine has a variety of uses. As a monomer dopamine can be used as a neurochemical to interact with an organism's neurochemistry to potentially influence the organism's health or disease pathogenesis. For example, dopamine has been used to treat issues, and associated symptoms, including, but not limited to, depression, the immune response, inflammation, gastric ulcers, and used as an intermediate for other neurochemicals. Additionally, it has been discovered that dopamine may be polymerized into dopamine polymers. These polymers also have multiple uses in many different disciplines. For example, dopamine polymers are being used in nanomedicine as cores for nanoparticles due to their strong adhesive properties, functionalization, and biocompatibility. Further, these polymers are used as a surface coating for dozens of different substrates with applications for consumer goods, biomedicine, energy, industrial, and military sectors. For example, dopamine polymers due to their even coating ability can be used in batteries to prevent side reactions that may shorten the batteries life. Because of all the different possible uses, the demand for large quantities of dopamine, either as a monomer or polymer, is increasing.

Dopamine is produced in vivo by the decarboxylation of L-DOPA, which is also referred to as levodopa and L-3,4-dihydroxyphenylalanie, a reaction usually catalyzed by an enzyme. These enzymes belong to the family of aromatic L-amino acid decarboxylases which includes tyrosine decarboxylase and histidine decarboxylase. These enzymes vary across species in both their substrate preference and their catalytic ability. Therefore, their use in industrial production remains to be resolved.

Industrial production of dopamine has been largely limited to non-biological chemical synthetic processes and biological synthesis using enzymatic systems that use tyrosine phenollyase, which produces L-DOPA. This provides a two-step reaction for dopamine synthesis from catechol, pyruvate, or ammonia into L-DOPA and thereafter dopamine as dopamine production requires a second enzyme for synthesis. There remains a need for more efficient methods for the synthesis of the neurochemical.

Accordingly, it is an objective of the invention to screen and identify microorganisms based on the nucleic acid or protein sequence of the aromatic decarboxylases capable of producing dopamine.

Another objective is to transfect or transform other cells with an aromatic decarboxylase capable of producing dopamine to confer or enhance their dopamine production.

It is a further objective of the invention to screen and identify high dopamine producing microorganisms which grow well in media to produce dopamine for industrial use. Further, it is an objective of the invention to provide kits and systems for the screening and identification of the high dopamine producing microorganisms.

Another objective of the claimed invention is to develop methods to produce dopamine for industrial use.

Other objects, advantages and features of the present invention will become apparent from the following specification taken in conjunction with the accompanying figures.

### BRIEF SUMMARY OF THE INVENTION

An advantage of the invention is the ability to screen and identify which bacterial strains are capable of producing dopamine, including for industrial use. It is an advantage of the present invention that certain bacteria, including certain strains of Enterococcus spp. are capable of producing dopamine in the gastrointestinal tract of an animal and/or human as well as in media mimicking the gastrointestinal tract and commercially available media. Such bacterial strains are advantageously combined with L-DOPA to produce dopamine either in vivo or in vitro.

Another advantage of the invention is the ability to produce dopamine with little or no oxidation.

In an embodiment, the present invention provides a method for selecting or identifying a microorganism capable of producing dopamine. In a further embodiment, the microorganism is identified by one or more nucleic acid sequences encoding an aromatic decarboxylase.

In further aspects, the microorganism may produce dopamine in the gut of a subject, media, or both.

In another embodiment, the present invention provides a method for enzymatically producing dopamine, comprised of providing a bacterial culture having an aromatic decarboxylase capable of producing dopamine with L-DOPA.

In another embodiment, the present invention provides a method for enhancing or conferring to a cell the ability to produce dopamine. In a further embodiment, said cell is prokaryotic. In another embodiment, said cell is eukaryotic.

In one aspect of the invention, the invention comprises a nucleic acid (a) encoding an aromatic decarboxylase enzyme that may be capable of L-DOPA decarboxylation in order to produce dopamine; or (b) having a nucleic acid sequence of SEQ ID NOS: 1, 2, 3, 4, 5, 6, 7, 8, 9, 10, 11, or 12; or (c) having a nucleic acid sequence which is at least 90% homology, more preferably at least 95% homology, or more preferably at least 96%, 97%, 98%, or 99% homology to one of SEQ ID NOS: 1, 2, 3, 4, 5, 6, 7, 8, 9, 10, 11, or 12; or (d) which hybridizes to a nucleic acid sequence which encodes the same under at least moderately stringent conditions.

In one aspect of the invention, the invention comprises aromatic decarboxylase proteins or peptides which may be capable of decarboxylating L-DOPA to produce dopamine, as well as modified forms, subsequences or fragments thereof. In one embodiment, the invention includes an aromatic decarboxylase polypeptide comprising (a) a polypeptide comprising at least 90% homology, more preferably at least 95% homology, or more preferably at least 96%, 97%, 98%, or 99% sequence identity to a polypeptide of SEQ ID NOS: 13, 14, 15, 16, 17, 18, 19, 20, 21, 22, 23, or 24 (b) a polypeptide encoded by a nucleic acid of the present invention; or (c) a polypeptide comprising L-DOPA decarboxylase activity and comprising at least 50 amino acids conserved of (a).

Amino acid sequences which are substantially similar to the amino acid sequences described above, and which are capable of decarboxylating L-DOPA are within the scope of this invention.

In another aspect of the invention, the invention comprises an expression vector comprising a nucleic acid sequence according to any one of the nucleic acids described above in functional combination with an expressible promoter.

In another aspect of the invention, the invention comprises a genetically modified cell transformed with the expression vector described above, wherein cell is modified in its dopamine production ability. In one embodiment, the cell is engineered to express the peptides of the invention and thus increases dopamine production. Non-genetic engineering methods of introducing the peptide to the cell are also contemplated.

In another aspect of the invention, the invention comprises a method for producing a genetically modified cell that has improved dopamine production comprising the steps of: a) introducing into a cell the expression vector as described above to produce a transformed cell; and b) culturing a transgenic/transformed cell from the transformed cell, wherein the transgenic culture has improved dopamine production compared to a nonmodified culture. In one embodiment, the transgenic cell is an Enterococcus spp.

In another embodiment of the invention, more than one gene or nucleic acid is transformed into a cell to further increase the production of dopamine.

In another aspect of the invention, the invention comprises an immunoassay method to detect dopamine production in cells by screening the same for an aromatic decarboxylase peptide of the invention of a nucleic acid molecule encoding the same. In another aspect of the invention, the invention comprises a polymerase chain reaction method to detect dopamine production in cells by screening the same for nucleic acid molecules of the invention. In yet another aspect of the invention, the invention comprises a strand displacement method to detect dopamine production in cells by screening the same for nucleic acid molecules of the invention.

Nucleotide sequences encoding the synthetic proteins disclosed herein can be used in developing other transgenic cells, vectors, antibodies and the like that can be routinely used in culturing cells for dopamine production.

In another aspect of the invention, the invention comprises kits and systems to identify and screen microorganisms which may produce dopamine.

The foregoing summary is illustrative only and is not intended to be in any way limiting. In addition to the illustrative aspects, embodiments, and features described above, further aspects, embodiments, and features will become apparent by reference to the drawings and the following detailed description. Accordingly, the drawings and detailed description are to be regarded as illustrative in nature and not restrictive.

### BRIEF DESCRIPTION OF THE DRAWINGS

FIG. 1A is a graphical representation of a control sample (medium alone) and FIG. 1B is a graphical representation of E. faecium producing dopamine from L-DOPA in simulated small intestine medium (sSIM) which shows the evaluation of an E. faecium probiotic in comparison to a control (medium alone) to assess the ability to produce dopamine utilizing a precursor L-DOPA in medium simulating gastrointestinal conditions according to embodiments of the invention.

FIG. 2A is a graphical representation showing the chromatogram of the control sSIM demonstrating no conversion of L-DOPA conversion without a monoculture of E. faecium in sSIM supplemented with 1.0×10⁻³ M L-DOPA. FIG. 2B is a graphical representation of strain ML1087 grown in 1.0×10⁻³ M L-DOPA supplemented sSIM. FIG. 2C is a graphical representation of ML1088 grown in 1.0×10⁻³ M L-DOPA supplemented sSIM. FIG. 2D is a graphical representation of ML1085 grown in 1.0×10⁻³ M L-DOPA supplemented sSIM. FIG. 2E is a graphical representation of ML1089 grown in 1.0×10⁻³ M L-DOPA supplemented sSIM. FIG. 2F is a graphical representation of ML1086 grown in 1.0×10⁻³ M L-DOPA supplemented sSIM. FIG. 2G is a graphical representation of ML1081 grown in 1.0×10⁻³ M L-DOPA supplemented sSIM. FIG. 2H is a graphical representation of ML1082 grown in 1.0×10⁻³ M L-DOPA supplemented sSIM.

FIG. 3 shows the dopamine production of an E. faecium strain, ML1082, demonstrated dose-dependent inhibition in the presence of the L-DOPA decarboxylase inhibitor Carbidopa.

FIG. 4A is a graphical representation showing the chromatogram of the conversion of L-DOPA into dopamine by E. faecium co-cultured in MRS media with Escherichia coli (E. coli) with and without carbidopa or deoxypyridoxine phosphate. FIG. 4B is a graphical representation showing the chromatogram of salsolinol produced by E. coli co-cultured in MRS media with E. faecium with and without carbidopa or deoxypyridoxine phosphate.

FIG. 5A is a graphical representation of the levels of dopamine and L-DOPA for multiple strains of E. faecium grown in BHI broth supplemented with L-DOPA compared to an uninoculated control. FIG. 5B is a graphical representation of the levels of dopamine and L-DOPA for multiple strains of E. faecium grown in Tryptic soy broth supplemented with L-DOPA compared to an uninoculated control. FIG. 5C is a graphical representation of the levels of dopamine and L-DOPA for multiple strains of E. faecium grown in MRS broth supplemented with L-DOPA compared to an uninoculated control. FIG. 5D is a graphical representation of the levels of dopamine and L-DOPA for multiple strains of E. faecium grown in Nutrient Broth supplemented with L-DOPA compared to an uninoculated control. FIG. 5E is a graphical representation of the levels of dopamine and L-DOPA for multiple strains of E. faecium grown in Peptone water supplemented with L-DOPA compared to an uninoculated control. FIG. 5F is a graphical representation of the levels of dopamine and L-DOPA for multiple strains of E. faecium grown in Luria broth supplemented with L-DOPA compared to an uninoculated control.

FIG. 6A is a graphical representation of a combination chromatograph which examines a dopamine producing culture for oxidized species of dopamine showing there is minimal overlap between peaks unique to oxidation and peaks generated during culture. FIG. 6B is a graphical representation of E. faecium cultured with 3.8 mM L-DOPA in MRS showing the change in pH over 12 hours with measurements taken every 15 minutes

FIG. 7 is a multiple sequence alignment of the amino acid sequence encoding the putative aromatic decarboxylases of six strains of Enterococcus faecium compared to tyrosine decarboxylase of Lactobacillus brevis and Streptococcus faecalis.

FIG. 8A is a graphical representation of the levels of L-DOPA and tyrosine in an uninoculated control sample of 1 mM L-DOPA containing sSIM showing no conversion of either L-DOPA or tyrosine into dopamine or tyramine. FIG. 8B is a graphical representation of the levels of L-DOPA and tyrosine in 1 mM L-DOPA containing sSIM inoculated with untransformed, wild type E. coli showing that the wild type E. coli is unable to convert L-DOPA and tyrosine into dopamine or tyramine. FIG. 8C is a graphical representation of the levels of L-DOPA and tyrosine of 1 mM L-DOPA containing sSIM inoculated with tyrDC/tryP transformed E. coli showing the conversion of L-DOPA and tyrosine into dopamine and tyramine.

### DETAILED DESCRIPTION OF THE INVENTION

The present invention relates to methods for selecting or identifying microorganisms capable of producing dopamine in the gut of an animal or human, media, or both through an aromatic decarboxylase capable of decarboxylating L-DOPA to produce dopamine. The present invention further relates to compositions comprising a nucleic acid encoding an aromatic decarboxylase capable of decarboxylating L-DOPA to produce dopamine. Still further, the present invention relates to cells which have improved or conferred dopamine production through the introduction of compositions comprising a nucleic acid encoding an aromatic decarboxylase capable of decarboxylating L-DOPA to produce dopamine when compared to cells lacking the aromatic decarboxylase, and the methods to make such. The present methods and compositions have many advantages over conventional administration and/or screening of microorganisms. Without being limited to the particular mechanisms and benefits of the invention, the methods and compositions overcome a lack of knowledge in ability to identify, select, and use microorganisms to produce dopamine based on a desirable mechanism of action, namely a microbial aromatic decarboxylase which can both bind to and decarboxylate L-DOPA to produce dopamine. The present invention overcomes these limitations and provides methods for selecting probiotic strains based upon the nucleic acids encoding such proteins, namely one or more of aromatic L-amino acid decarboxylase, such as a tyrosine decarboxylase and/or histidine decarboxylase.

The embodiments of this invention are not limited to particular methods of selection, methods of production, and compositions, which can vary and may be understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Definitions

In order to provide a clear and consistent understanding of the specification and the claims, including the scope given to such terms, the following definitions are provided. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence-based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D. C. (1993). The product of amplification is termed an amplicon.

The term “conservatively modified variants” applies to both amino acid and nucleic acid sequences. With respect to particular nucleic acid sequences, a conservatively modified variant refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate.

Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W). See also, Creighton (1984) Proteins W. H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)).

The term “nucleic acid construct” or “polynucleotide construct” means a nucleic acid molecule, either single-stranded or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

The term “control sequences” is defined herein to include all components, which are necessary or advantageous for the expression of a polypeptide of the present invention. Each control sequence may be native or foreign to the nucleotide sequence encoding the polypeptide. Such control sequences include, but are not limited to, a leader sequence, polyadenylation sequence, propeptide sequence, promoter sequence, signal peptide sequence, and transcription terminator sequence. At a minimum, the control sequences include a promoter and transcriptional and translational stop signals. The control sequences may be provided with linkers for the purpose of introducing specific restriction sites facilitating ligation of the control sequences with the coding region of the nucleotide sequence encoding a polypeptide.

When used herein the term “coding sequence” is intended to cover a nucleotide sequence, which directly specifies the amino acid sequence of its protein product. The boundaries of the coding sequence are generally determined by an open reading frame, which usually begins with the ATG start codon. The coding sequence typically includes a DNA, cDNA, and/or recombinant nucleotide sequence.

In the present context, the term “expression” includes any step involved in the production of the polypeptide including, but not limited to, transcription, post-transcriptional modification, translation, post-translational modification, and secretion.

In the present context, the term “expression vector” covers a DNA molecule, linear or circular, that comprises a segment encoding a polypeptide of the invention, and which is operably linked to additional segments that provide for its transcription.

The term “host cell”, as used herein, includes any cell type which is susceptible to transformation with a nucleic acid construct. Host cells may be prokaryotic cells such as, but not limited to, E. coli, or eukaryotic cells such as, but not limited to, yeast, mycobiome, insect, amphibian, or mammalian cells. Preferably, host cells are bacterial.

The term “cell”, as used herein, includes any cell type and may be prokaryotic cells, such as, but not limited, to E. coli or E. faecium, or eukaryotic cells, such as, but not limited to, yeast, mycobiome, insect, amphibian, or mammalian cells. Preferably, cells contain an aromatic decarboxylase which may convert L-DOPA into dopamine.

The term “effective dose” (or “effective amount”) refers to an amount of an active ingredient, e.g., an inhibitor according to the invention, sufficient to effect beneficial or desired results when administered to a subject or contacted to a cell. An effective dose can be administered or applied in one or more administrations, applications or dosages. An effective dose of a composition according to the invention may be readily determined by one of ordinary skill in the art.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g., a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous

Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or cDNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning--A Laboratory Manual, 2nd ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. As used herein, “polynucleotide” or includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof.

Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of post-translation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “bacterial promoter” is a promoter capable of initiating transcription in bacterial cells whether or not its origin is a bacterial cell. Exemplary bacterial promoters include, but are not limited to, those that are obtained from bacteria, bacterial viruses, and eukaryotes which comprise genes expressed in bacterial cells such as yeast. Examples of promoters under developmental control include promoters that preferentially initiate transcription in certain tissues, such as the gut. Such promoters are referred to as “tissue preferred”. Promoters which initiate transcription only in certain tissue are referred to as “tissue specific”. A “cell type” specific promoter primarily drives expression in certain cell types in one or more organs, for example, the small intestine of the alimentary track. An “inducible” or “repressible” promoter is a promoter which is under environmental control. Examples of environmental conditions that may affect transcription by inducible promoters include anaerobic conditions or the presence of neurochemical precursor. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids.

Selectively hybridizing sequences typically have about at least 80% sequence identity, preferably 90% sequence identity, and most preferably 100% sequence identity (i.e., complementary) with each other.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1× SSC at 55 to 50° C. Exemplary high stringency conditions include hybridization in 50% formamide, 1 M NaCl, 1% SDS at 37° C., and a wash in 0.1× SSC at 60 to 65° C. for 20 minutes.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C. +16.6 (log M)+0.41 (% GC)−0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tm is reduced by about 1° C. for each 1% of mismatching; thus, Tₘ, hybridization and/or wash conditions can be adjusted to hybridize to sequences of the desired identity. For example, if sequences with ≥90% identity are sought, the Tm can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology-Hybridization with Nucleic Acids Probes, Part I, Chapter 2, Ausubel, et al., Eds., Greene Publishing and Wiley-Interscience, New York (1995). In general, a high stringency wash is 2× 15 min in 0.5× SSC containing 0.1% SDS at 65° C.

As used herein, “genetically modified cell” includes reference to a cell which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional culture methods or by naturally occurring events such as random cross-fertilization, non-recombinant viral infection, non-recombinant bacterial transformation, non-recombinant transposition, or spontaneous mutation. As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of p Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=-4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

The term “about,” as used herein, refers to variations in size, distance or any other types of measurements that can be resulted from inherent heterogeneous nature of the measured objects and imprecise nature of the measurements itself. The term “about” also encompasses variation in the numerical quantity that can occur, for example, through typical measuring and liquid handling procedures used for making media and reagents; through inadvertent error in these procedures; through differences in the manufacture, source, or purity of the ingredients used to make the compositions or carry out the methods, and moreover may modify the typical measurements referenced herein, and the like. Whether or not modified by the term “about”, the claims include equivalents to the quantities.

As used herein “antibodies” and like terms refer to immunoglobulin molecules and immunologically active portions of immunoglobulin (Ig) molecules, i.e., molecules that contain an antigen binding site that specifically binds (immunoreacts with) an antigen. These include, but are not limited to, polyclonal, monoclonal, chimeric, single chain, Fc, Fab, Fab′, and Fab2 fragments, and a Fab expression library. Antibody molecules relate to any of the classes IgG, IgM, IgA, IgE, and IgD, which differ from one another by the nature of heavy chain present in the molecule. These include subclasses as well, such as IgG1, IgG2, and others. The light chain may be a kappa chain or a lambda chain. Reference herein to antibodies includes a reference to all classes, subclasses, and types. Also included are chimeric antibodies, for example, monoclonal antibodies or fragments thereof that are specific to more than one source, e.g., a mouse or human sequence.

The term “kit” as used herein refers to a set of reagents for the purpose of performing the method of the invention, more particularly, the identification of aromatic decarboxylases.

As used herein, the “alimentary tract” refers to the pathway by which food enters the body of a subject, is digestively processed and ultimately expelled as solid waste. The alimentary canal includes, for example, the mouth, pharynx, esophagus, stomach, small intestine, large intestine, and anus.

Also, as used herein, the term “gut” refers to the gastrointestinal tract as well as the liver, spleen, pancreas, omentum, and other organs served by the blood supply to and from the gut.

The term “intestinal microbiota”, as used herein, refers to the population of microorganisms inhabiting the gastrointestinal tract. The term was previously referred to as the intestinal flora.

The term “microbiome”, as used herein, refers to a population of microorganisms from a particular environment, including the environment of the body or a part of the body. The term is interchangeably used to address the population of microorganisms itself (sometimes referred to as the microbiota), as well as the collective genomes of the microorganisms that reside in the particular environment. The term “environment,” as used herein, refers to all surrounding circumstances, conditions, or influences to which a population of microorganisms is exposed. The term is intended to include environments in a subject, such as a human and/or animal subj ect.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. The term “microbial” indicates pertaining to, or characteristic of a microorganism.

As used herein, the term “neurochemical” refers to small organic molecules and peptides that participate in neural, immune and other general physiological activities. Neurochemicals can be produced within in various parts of a subject, such as the gut, brain, etc. Such biogenic neurochemicals are capable of eliciting neural activity. Exemplary neurochemicals include both neurotransmitters and neuromodulators, which can be either excitatory or inhibitor in nature. Exemplary neurochemicals include catecholamines. Further exemplary neurochemicals include glutamate, dopamine, serotonin, histamine, norepinephrine, epinephrine, phenethylamines, thyronamine compounds, tryptamine, GABA, acetylcholine, and the like.

As used herein, the term “aromatic decarboxylase” and “aromatic decarboxylases” is used interchangeably with “aromatic L-amino acid decarboxylase” and “aromatic L-amino acid decarboxylases,” respectively.

“Non-pathogenic bacteria” refers to bacteria that under normal conditions do not cause a disease or harmful responses in a healthy host. In some embodiments, non-pathogenic bacteria are commensal bacteria. Examples of non-pathogenic bacteria include, but are not limited to Bacillus spp., Bacteroides spp Bifidobacterium spp., Brevibacteria spp., Clostridium spp., Enterococcus spp., Escherichia coli, Lactobacillus spp., Lactococcus spp., Saccharomyces spp., and Staphylococcus spp. Naturally pathogenic bacteria may be genetically engineered to provide reduced or eliminate pathogenicity according to standard methods in the art.

Non-pathogenic bacteria and/or yeast may be genetically engineered to enhance or improve desired biological properties, e.g., survivability. Non-pathogenic bacteria and/or yeast may be genetically engineered to provide probiotic properties. Bacteria and/or yeast may be genetically engineered to be non-pathogenic. Without being limited to a particular mechanism of the invention, probiotics differ in their ability to produce neurochemicals in the gut of a subject. Non-pathogenic bacteria may be used for probiotic or synbiotic compositions used to treat subjects, while either pathogenic or non-pathogenic bacteria may be used for production of dopamine in a bioreactor or media. Pathogenicity, or virulence, of E. faecium may be defined as in the European Food Safety Authority, Scientific Opinion on the safety and efficacy of Oralin® (Enterococcus faecium) as a feed additive for calves for rearing, piglets, chickens for fattening, turkeys for fattening and dogs, EFSA Journal 2014;12(6):3727, 19 pp. (doi:10.2903/j.efsa.2014.3727) in section 2.1.1.

The term “population”, as used herein, refers to a plurality of individual organisms, in the context of this invention, the term refers in particular to a collection of organisms of diverse taxonomic affiliation, in particular bacteria.

“Prebiotic” is used to refer to a food or dietary supplement that is associated with modulating a microbiota thus conferring a health benefit on a subject. Prebiotics in most instances are not drugs, functioning due to changes to the resident bacteria either changing the proportions of the resident bacteria or the activities thereof and not functioning because of absorption of the component or due to the component acting directly on the subject. As referred to herein, a prebiotic includes a precursor and/or co-factor to a neurochemical for combined use with a probiotic.

“Probiotic” is used to refer to live, non-pathogenic microorganisms, e.g., bacteria or fungi, which can confer health benefits to a host organism that contains an appropriate amount of the microorganism. In some embodiments, the host organism is a mammal. In some embodiments, the host organism is a human. Some species, strains, and/or subtypes of non-pathogenic bacteria and yeast are currently recognized as probiotics. Examples of probiotics include, but are not limited to, Candida spp., Debaryomyces spp., Debaryomyces spp., Enterococcus spp., Kluyveromyces spp., Kluyveromyces spp., Saccharomyces spp., Yarrowia spp., Bifidobacteria spp., Escherichia coli, Vagococcus spp., Carnobacterium spp., Melissococcus spp. and Lactobacillus spp., e.g., Candida humilis, Debaryomyces hansenii, Debaryomyces occidentalis, Kluyveromyces lactis, Kluyveromyces lodderae, Kluyveromyces marxianus, Saccharomyces cerevisiae, Saccharomyces boulardii, Yarrowia lipolytica, Bifidobacterium bifidum, Enterococcus faecium, Enterococcus faecalis, Enterococcus hirae, Enterococcus casseliflavus, Enterococcus gallinarum, Escherichia coli strain Nissle, Lactobacillus acidophilus, Lactobacillus bulgaricus, Lactobacillus paracasei, Lactobacillus plantarum, Vagococcus fluvaialis (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006).

The term “sample,” as used herein, refers to any sample suitable for analyzing or typing according to the methods of the present invention. A sample may be collected from an organism (e.g., human or other mammal subject) and can be in any form, including without limitation a solid material such as a tissue, cells, a cell pellet, a cell extract, or a biopsy, or a biological fluid such as urine, blood, stool, saliva, amniotic fluid, exudate from a region of infection or inflammation, or the like. A sample may be collected from a culture, including without limitation from the media or a subset of cells from the culture's population. The term “sufficient amount of time,” as used herein, refers to the time it takes for a compound, material, composition comprising a compound of the present invention, or an organism which is effective for producing some desired effect in at least a sub-population of cells.

As used herein, “substantially free” may refer to any component that the composition of the invention lacks or mostly lacks. When referring to “substantially free” it is intended that the component is not intentionally added to compositions of the invention. Use of the term “substantially free” of a component allows for trace amounts of that component to be included in compositions of the invention because they are present in another component. However, it is recognized that only trace or de minimus amounts of a component will be allowed when the compositions is said to be “substantially free” of that component. Moreover, the term if a composition is said to be “substantially free” of a component, if the component is present in trace or de minimus amounts it is understood that it will not affect the effectiveness of the compositions. It is understood that if an ingredient is not expressly included herein or its possible inclusion is not stated herein, the invention composition may be substantially free of that ingredient. Likewise, the express inclusion of an ingredient allows for its express exclusion thereby allowing a composition to be substantially free of that expressly stated ingredient.

The present invention provides, inter alia, compositions and methods for the production of dopamine. The compositions of the invention relate to the identification of dopamine production genes identified from Enterococcus spp. Thus, these genes may be used to confer or improve dopamine production in other organisms, such as other bacteria, yeast, insects, plants, or mammalian cells. Thus, the genes and proteins identified here may be introduced or modulated to confer improved dopamine production to other organisms. These genes, nucleic acid sequences or proteins can be transferred into organisms to confer or improve dopamine production, can be modified to engineer gene sequences or amino acid sequences for broader catecholamine production, or can be used to isolate and identify alternate gene forms and markers which may be used in cultures for large scale production of dopamine. By “confer or improve dopamine production” it is intended that the proteins or sequences, either alone or in combination with other proteins or sequences, enhance the production of dopamine within a population of cells. In this manner, dopamine production can be enhanced or improved in the transformed cell or its progeny when at least one of the sequences of the invention is introduced to a single cell or otherwise modulated according to the invention.

The compositions include nucleic acid molecules comprising sequences of bacterial genes and the polypeptides encoded thereby which are associated with the production of dopamine from L-DOPA have been identified. Particularly, the nucleotide and amino acid molecules of putative Enterococcus faecium (SEQ ID NOS: 1, 2, 3, 4, 5, 6, 7, 8, 9, 10, 11, 12, 13, 14, 15, 16, 17, 18, 19, 20, 21, 22, 23, and 24) and any conservatively modified variants, fragments, and homologs or full-length sequences incorporating the same which retain the L-DOPA decarboxylase related activity described herein are part of the invention. The compositions may comprise a nucleic acid (a) encoding an aromatic decarboxylase enzyme that may be capable of L-DOPA decarboxylation in order to produce dopamine; or (b) having a nucleic acid sequence of SEQ ID NOS: 1, 2, 3, 4, 5, 6, 7, 8, 9, 10, 11, or 12; or (c) having a nucleic acid sequence which is at least 90% homology, more preferably at least 95% homology, or more preferably at least 96%, 97%, 98%, or 99% homology to one of SEQ ID NOS: 1, 2, 3, 4, 5, 6, 7, 8, 9, 10, 11, or 12; or (d) which hybridizes to a nucleic acid sequence which encodes the same under at least moderately stringent conditions. The compositions may comprise a aromatic decarboxylase proteins or peptides which may be capable of decarboxylating L-DOPA to produce dopamine, as well as modified forms, subsequences or fragments thereof. In one embodiment, the invention includes a aromatic decarboxylase polypeptide comprising (a) a polypeptide comprising at least 90% homology, more preferably at least 95% homology, or more preferably at least 96%, 97%, 98%, or 99% sequence identity to a polypeptide of SEQ ID NOS: 13, 14, 15, 16, 17, 18, 19, 20, 21, 22, 23, or 24 (b) a polypeptide encoded by a nucleic acid of the present invention; or (c) a polypeptide comprising L-DOPA decarboxylase activity and comprising at least 50 amino acids conserved of (a).

Promoter and other regulatory elements which are natively associated with these genes can be easily isolated using the sequences and methods described herein with no more than routine experimentation. These sequences can also be used to identify promoters, enhancers or other signaling sequences in the regulatory regions of resistance genes. Such regulatory elements or promoters would provide for temporal and spatial expression of operably linked sequences with dopamine production in a microorganism. Nucleotide sequences operably linked to such promoter sequences are transformed into a cell. Exposure of the transformed cell to L-DOPA could induce transcriptional activation of the nucleotide sequences operably linked to these promoter regulatory sequences.

The compositions and methods of the invention are presumably involved in biochemical pathways and as such may also find use in the activation or modulation of expression of other genes, including those involved in other aspects of L-DOPA uptake into the cell or dopamine export out of the cell.

By “modulating” or “modulation” it is intended that the level of expression of a gene may be increased or decreased relative to genes driven by other promoters or relative to the normal or uninduced level of the gene in question.

The present invention provides for isolated nucleic acid molecules comprising nucleotide molecules encoding the amino acid sequence herein: putative Enterococcus faecium aromatic decarboxylase (SEQ ID NOS: 1, 2, 3, 4, 5, 6, 7, 8, 9, 10, 11, 12, 13, 14, 15, 16, 17, 18, 19, 20, 21, 22, 23, and 24) and any conservatively modified variants, fragments, and homologs or full length sequences incorporating the same which retain the L-DOPA decarboxylase activity described herein. Further provided are polypeptides having an amino acid sequence encoded by a nucleic acid molecule described herein, for example those polypeptides comprising the sequences set forth in the herein, and fragments and variants thereof. The compositions may comprise a nucleic acid (a) encoding an aromatic decarboxylase enzyme may be capable of L-DOPA decarboxylation in order to produce dopamine; or (b) having a nucleic acid sequence of SEQ ID NOS: 1, 2, 3, 4, 5, 6, 7, 8, 9, 10, 11, or 12; or (c) having a nucleic acid sequence which is at least 90% homology, more preferably at least 95% homology, or more preferably at least 96%, 97%, 98%, or 99% homology to one of SEQ ID NOS: 1, 2, 3, 4, 5, 6, 7, 8, 9, 10, 11, or 12; or (d) which hybridizes to a nucleic acid sequence which encodes the same under at least moderately stringent conditions. The compositions may comprise an aromatic decarboxylase proteins or peptides which may be capable of decarboxylating L-DOPA to produce dopamine, as well as modified forms, subsequences or fragments thereof. In one embodiment, the invention includes an aromatic decarboxylase polypeptide comprising (a) a polypeptide comprising at least 90% homology, more preferably at least 95% homology, or more preferably at least 96%, 97%, 98%, or 99% sequence identity to a polypeptide of SEQ ID NOS: 13, 14, 15, 16, 17, 18, 19, 20, 21, 22, 23, or 24 (b) a polypeptide encoded by a nucleic acid of the present invention; or (c) a polypeptide comprising L-DOPA decarboxylase activity and comprising at least 50 amino acids conserved of (a).

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In some embodiments, an “isolated” nucleic acid is free of sequences (such as other protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 20 kb, 10 kb, 5 kb, 4 kb, 3 kb, 2 kb, 1 kb, 0.5 kb, 0.4 kb, 0.3 kb, 0.2 kb, or 0.1 kb, or 50, 40, 30, 20 or 10 nucleotides that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, culture medium may represent less than about 30%, 20%, 10%, or 5% (by dry weight) of chemical precursors or non-protein-of-interest chemicals.

Fragments and variants of the disclosed nucleotide sequences are encompassed by the present invention. Fragments and variants of proteins encoded by the disclosed nucleotide sequences are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence affect dopamine production by retaining decarboxylase activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 75 nucleotides, about 100 nucleotides, and up to the full-length nucleotide sequence encoding the proteins of the invention.

A fragment of a non-host resistance nucleotide sequence that encodes a biologically active portion of a non-host resistance protein of the invention will encode at least 12, 25, 30, 50, 75, etc. contiguous amino acids, or up to the total number of amino acids present in a full-length non-host resistance protein of the invention.

Fragments of an aromatic decarboxylase nucleotide sequence that are useful as hybridization probes or PCR primers generally may or may not encode a biologically active portion of a protein. Thus, a fragment of an aromatic decarboxylase protein nucleotide sequence may encode a biologically active portion of an aromatic decarboxylase protein, or it may be used as a hybridization probe or PCR primer using methods disclosed below. A biologically active portion of an aromatic decarboxylase protein can be prepared by isolating a portion of the non-host resistance nucleotide sequences of the invention, expressing the encoded portion of the aromatic decarboxylase protein (e.g., by recombinant expression in vitro), and assessing the activity of the encoded portion of the non-host resistance protein. Nucleic acid molecules that are fragments of an aromatic decarboxylase nucleotide sequence comprise at least 16, 20, 25, 30, 40, 50, 60, 70, 80, 90, 100, 125, 150, 175, 200, etc. nucleotides, or up to the number of nucleotides present in a full-length non-host resistance nucleotide sequences disclosed herein.

The proteins of the invention may be altered in various ways including amino acid substitutions, deletions, truncations, and insertions. Methods for such manipulations are generally known in the art. For example, amino acid sequence variants of the aromatic decarboxylase proteins can be prepared by mutations in the DNA. Methods for mutagenesis and nucleotide sequence alterations are well known in the art. See, for example, Kunkel (1985) Proc. Nat. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be made.

It is recognized that having identified the nucleotide sequences disclosed herein, it is within the state of the art to isolate and identify regulatory elements in the 5′ untranslated region upstream from regions defined herein. Thus, for example, the promoter regions of the gene sequences disclosed herein may further comprise upstream regulatory elements that confer expression of heterologous nucleotide sequences operably linked to the disclosed promoter sequence. See particularly, Australian Patent No. AU-A-77751/94 and U.S. Pat. Nos. 5,466,785 and 5,635,618. It is also recognized by those of skill in the art that regulatory elements may be found in transcribed regions of a gene, for example in the region between transcription start and translation start as well as 3′ to the end of translation; such elements may be found in the sequences set forth herein.

The nucleotide sequences of the invention can be used to isolate corresponding sequences from other organisms, particularly other microorganisms, more particularly other bacteria. In this manner, methods such as PCR, hybridization, and the like can be used to identify such sequences based on their sequence homology to the sequences set forth herein. Sequences isolated based on their sequence identity to the nucleotide sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species. Thus, isolated sequences that have L-DOPA decarboxylase-like activity or and which hybridize under stringent conditions to the aromatic decarboxylase sequences disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press Plainview, New York). See also Innis et al., eds. (1990) PCR Protocols: A Guide to Methods and Applications (Academic Press, New York); Innis and

Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present it a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the aromatic decarboxylase sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, New York).

For example, an entire sequence disclosed herein, or one or more portions thereof, may be used as a probe capable of specifically hybridizing to corresponding aromatic decarboxylase sequences, including promoters and messenger RNAs. To achieve specific hybridization under a variety of conditions, such probes include sequences that are unique among aromatic decarboxylase sequences and may be at least about 10 or 15 or 17 nucleotides in length or at least about 20 or 22 or 25 nucleotides in length. Such probes may be used to amplify corresponding sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, New York).

Techniques that measure dopamine production induced by the signal pathway from the sequences herein are commonly known in the art, such as, but not limited to high performance liquid chromatography (HPLC) with either electrochemical detection or ultraviolet detection, immunoassays, and cyclic voltammetry. Within the art, there exist assays which may be used to measure the activity of the polypeptides of the invention. Such techniques include, measuring over time or measuring at a fixed time. For example, a bacterial colony expressing a dopamine producing polypeptide shows an increase in the concentration of dopamine in the culture media when compared to a control bacterial colony that does not express the dopamine producing composition.

The compositions and methods of the invention function to increase the production of dopamine. The gene products may accomplish their increased production effects by preferentially binding to L-DOPA or increase the decarboxylation rate of L-DOPA.

The cells that have been transformed may be grown into populations or grown into plants or animals in accordance with conventional ways. For example, transformed bacteria or yeast may be cultured in the appropriate media. Such media could include, for example, Luria broth, MRS, BHI, tryptic soy broth, nutrient broth, peptone water, or media that closely simulates the environment in the gastrointestinal tract. The culture of cell lines for animals and plants is also contemplated, and cell lines and media for each are known in the art. For plants, see, for example, McCormick et al. (1986) Plant Cell Reports 5:8184. For plants, they may then be grown, and either pollinated with the same transformed strain or different strains, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

The invention in one aspect comprises expression constructs comprising a DNA sequence which encodes upon expression an aromatic decarboxylase nucleic acid sequence operably linked to a promoter to direct expression of the protein. These constructs are then introduced into host cells using standard molecular biology techniques. The invention can be also be used for hybrid animal or plant or seed production, once transgenic inbred parental lines have been established.

The methods of the invention described herein may be applicable to any species of prokaryote or eukaryotic host cell.

Production of a genetically modified host cell either expressing or inhibiting expression of a structural gene combines the teachings of the present disclosure with a variety of techniques and expedients known in the art. In most instances, alternate expedients exist for each stage of the overall process. The choice of expedients depends on the variables such as the plasmid vector system chosen for the cloning and introduction of the recombinant DNA molecule, the host cell to be modified, the particular structural gene, promoter elements and upstream elements used. Persons skilled in the art are able to select and use appropriate alternatives to achieve functionality. Culture conditions for expressing desired structural genes and cultured cells are known in the art. Truncated promoter selection and structural gene selection are other parameters which may be optimized to achieve desired host cell expression or inhibition as is known to those of skill in the art and taught herein.

The following is a non-limiting general overview of Molecular biology techniques which may be used in performing the methods of the invention.

### Promoters

The constructs, promoters or control systems used in the methods of the invention may include a bacterial promoter, a tissue specific promoter, an inducible promoter or a constitutive promoter.

A large number of suitable promoter systems are available. For example, one constitutive promoter useful for the invention is the cauliflower mosaic virus (CaMV) 35S. It has been shown to be highly active in many plant organs and during many stages of development when integrated into the genome of transgenic plants and has been shown to confer expression in protoplasts of both dicots and monocots.

Bacterial promoters may include, but are not limited to, the lad promoter, Tac promoter, or the T7 promoter.

Organ-specific promoters appropriate for a desired target organ can be isolated using known procedures. These control sequences are generally associated with genes uniquely expressed in the desired organ. For example, in a typical higher plant or animal, each organ has thousands of mRNAs that are absent from other organ systems (reviewed in Goldberg, Phil, Trans. R. Soc. London (1986) B314-343). mRNAs are first isolated to obtain suitable probes for retrieval of the appropriate genomic sequence which retains the presence of the natively associated control sequences. An example of the use of techniques to obtain the cDNA associated with mRNA specific to avocado fruit is found in Christoffersen et al., Plant Molecular Biology (1984) 3:385. Briefly, mRNA was isolated from ripening avocado fruit and used to make a cDNA library. Clones in the library were identified that hybridized with labeled RNA isolated from ripening avocado fruit, but that did not hybridize with labeled RNAs isolated from unripe avocado fruit. Many of these clones represent mRNAs encoded by genes that are transcriptionally activated at the onset of avocado fruit ripening.

Another very important method that can be used to identify cell type specific promoters that allow even to identification of genes expressed in a single cell is enhancer detection (O'Kane, C., and Gehring, W.J. (1987), “Detection in situ of genomic regulatory elements in Drosophila”, Proc. Natl. Acad. Sci. USA, 84, 9123-9127). This method was first developed in Drosophila and rapidly adapted to mice and plants (Wilson, C., Pearson, R. K., Bellen, H. J., O'Kane, C. J., Grossniklaus, U., and Gehring, W. J. (1989), “P-element-mediated enhancer detection: an efficient method for isolating and characterizing developmentally regulated genes in Drosophila”, Genes & Dev., 3, 1301-1313; Skarnes, W. C. (1990), “Entrapment vectors: a new tool for mammalian genetics”, Biotechnology, 8, 827-831; Topping, J. F., Wei, W., and Lindsey, K. (1991), “Functional tagging of regulatory elements in the plant genome”, Development, 112, 1009-1019; Sundaresan, V., Springer, P. S., Volpe, T., Haward, S., Jones, J. D. G., Dean, C., Ma, H., and Martienssen, R. A., (1995), “Patterns of gene action in plant development revealed by enhancer trap and gene trap transposable elements”, Genes & Dev., 9, 1797-1810).

The promoter used in the method of the invention may be an inducible promoter. An inducible promoter is a promoter that is capable of directly or indirectly activating transcription of a DNA sequence in response to an inducer. In the absence of an inducer, the DNA sequence will not be transcribed. Typically, the protein factor that binds specifically to an inducible promoter to activate transcription is present in an inactive form which is then directly or indirectly converted to the active form by the inducer. The inducer may be a chemical agent such as a protein, metabolite (sugar, alcohol etc.), a growth regulator, or a neurochemical precursor. A host cell containing an inducible promoter may be exposed to an inducer by externally applying the inducer to the cell such as by adding the inducer to a culture media, spraying, watering, heating, or similar methods. Examples of inducible promoters include the inducible 70 kd heat shock promoter of D. melanogaster (Freeling, M., Bennet, D. C., Maize ADN 1, Ann. Rev. of Genetics, 19:297-323) and the alcohol dehydrogenase promoter which is induced by ethanol (Nagao, R. T., et al., Miflin, B. J., Ed. Oxford Surveys of Plant Molecular and Cell Biology, Vol.

3, p. 384-438, Oxford University Press, Oxford 1986) or the Lex A promoter which is triggered with chemical treatment and is available through Ligand pharmaceuticals. The inducible promoter may be in an induced state throughout seed formation or at least for a period which corresponds to the transcription of the DNA sequence of the recombinant DNA molecule(s).

The preferred promoters may be used in conjunction with naturally occurring flanking coding or transcribed sequences.

It may also be desirable to include some intron sequences in the promoter constructs since the inclusion of intron sequences in the coding region may result in enhanced expression and specificity. Thus, it may be advantageous to join the DNA sequences to be expressed to a promoter sequence that contains the first intron and exon sequences of a polypeptide which is unique to cells/tissues of a plant critical to female gametophyte development and/or function.

Additionally, regions of one promoter may be joined to regions from a different promoter in order to obtain the desired promoter activity resulting in a chimeric promoter. Synthetic promoters which regulate gene expression may also be used.

The expression system may be further optimized by employing supplemental elements such as transcription terminators and/or enhancer elements.

### Other Regulatory Elements

In addition to a promoter sequence, an expression cassette or construct should also contain a transcription termination region downstream of the structural gene to provide for efficient termination. The termination region or polyadenylation signal may be obtained from the same gene as the promoter sequence or may be obtained from different genes. Polyadenylation sequences include, but are not limited to, the Agrobacterium octopine synthase signal (Gielen et al., EMBO J. (1984) 3:835-846) or the nopaline synthase signal (Depicker et al., Mol. and Appl. Genet. (1982) 1:561-573).

### Marker Genes

Recombinant DNA molecules containing any of the DNA sequences and promoters described herein may additionally contain selection marker genes which encode a selection gene product which confer on a host cell resistance to a chemical agent or physiological stress or confers a distinguishable phenotypic characteristic to the cells such that the host cells transformed with the recombinant DNA molecule may be easily selected using a selective agent. One such selection marker gene is neomycin phosphotransferase (NPT II) which confers resistance to kanamycin and the antibiotic G-418. Cells transformed with this selection marker gene may be selected for by assaying for the presence in vitro of phosphorylation of kanamycin using techniques described in the literature or by testing for the presence of the mRNA coding for the NPT II gene by Northern blot analysis in RNA from the tissue of the transformed cells. Polymerase chain reactions are also used to identify the presence of a transgene or expression using reverse transcriptase PCR amplification to monitor expression and PCR on genomic DNA. Other commonly used selection markers include the ampicillin resistance gene, the tetracycline resistance and the hygromycin resistance gene. Transformed plant cells thus selected can be induced to differentiate into plant structures which will eventually yield whole plants. It is to be understood that a selection marker gene may also be native to a plant.

### Transformation and Transfection

Numerous methods for transformation and transfection have been developed, including biological and physical. See, for example, Carson et al., Molecular Biology Techniques (Academic Press, London, 2012); Alberts et al., Molecular Biology of the Cell, 6^{th} ed., (Garland Science, Taylor & Francis Group, LLC, New York, 2015); and Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88. In addition, expression vectors and in vitro culture methods for cells or tissue transformation and regeneration of plants are available. See, for example, Carson et al., Molecular Biology Techniques (Academic Press, London, 2012), Alberts et al., Molecular Biology of the Cell, 6^{th} ed., (Garland Science, Taylor & Francis Group, LLC, New York, 2015) and Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

In addition to Agrobacterium transformation for plants, several methods of eukaryotic transformation, collectively referred to as direct gene transfer, have been developed. For example, a generally applicable method of transformation is microprojectile-mediated transformation wherein DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate cell walls and membranes. Sanford et al., Part. Sci. Technol. 5: 27 (1987), Sanford, J. C., Trends Biotech. 6: 299 (1988), Klein et al., Bio/Technology 6: 559-563 (1988), Sanford, J. C., Physiol Plant 79: 206 (1990), Klein et al., Biotechnology 10: 268 (1992). See also U.S. Pat. No. 5,015,580 (Christou, et al), issued May 14, 1991; U.S. Pat. No. 5,322,783 (Tomes, et al.), issued Jun. 21, 1994.

Another method for physical delivery of DNA to eukaryotic cells is sonication of target cells. Zhang et al., Bio/Technology 9: 996 (1991). Alternatively, liposome or spheroplast fusion have been used to introduce expression vectors into host cells. Deshayes et al., EMBO J., 4: 2731 (1985), Christou et al., Proc Natl. Acad. Sci. U.S.A. 84: 3962 (1987). Direct uptake of DNA into plant protoplasts using CaCl₂ precipitation, polyvinyl alcohol or poly-L-ornithine have also been reported. Hain et al., Mol. Gen. Genet. 199: 161 (1985) and Draper et al., Plant Cell Physiol. 23: 451 (1982). Electroporation of plant protoplasts and whole cells and tissues have also been described. Donn et al., In Abstracts of VII^{th} International Congress on Plant Cell and Tissue Culture IAPTC, A2-38, p 53 (1990); D'Halluin et al., Plant Cell 4: 1495-1505 (1992) and Spencer et al., Plant Mol. Biol. 24: 51-61 (1994).

Following transformation of target tissues, expression of the above-described selectable marker genes allows for preferential selection of transformed cells, tissues and/or plants, using regeneration and selection methods now well known in the art.

It is often desirable to have the DNA sequence in homozygous state which may require more than one transformation event to create a parental line in eukaryotic cells, requiring transformation with a first and second recombinant DNA molecule both of which encode the same gene product. It is further contemplated in some of the embodiments of the process of the invention that a plant cell be transformed with a recombinant DNA molecule containing at least two DNA sequences or be transformed with more than one recombinant DNA molecule. The DNA sequences or recombinant DNA molecules in such embodiments may be physically linked, by being in the same vector, or physically separate on different vectors. A cell may be simultaneously transformed with more than one vector provided that each vector has a unique selection marker gene. Alternatively, a cell may be transformed with more than one vector sequentially allowing an intermediate regeneration step after transformation with the first vector. Further, it may be possible to perform a sexual cross between individual sexually reproducing organism containing different DNA sequences or recombinant DNA molecules preferably the DNA sequences or the recombinant molecules are linked or located on the same chromosome, and then selecting from the progeny of the cross, plants containing both DNA sequences or recombinant DNA molecules.

Expression of recombinant DNA molecules containing the DNA sequences and promoters described herein in transformed plant cells may be monitored using Northern blot techniques and/or Southern blot techniques known to those of skill in the art.

The transformed cells may then be regenerated into a transgenic cellular population, plant, or animal.

After the expression or inhibition cassette is stably incorporated into a sexually reproducing organism, it can be transferred to other plants by sexual crossing. Any of a number of standard breeding techniques can be used, depending upon the species to be crossed.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA, 96:8774-8778; herein incorporated by reference.

### Molecular Markers

The present invention provides a method of genotyping a host cell comprising a heterologous polynucleotide of the present invention. Genotyping provides a means of distinguishing homologs of a chromosome pair and can be used to differentiate segregants in a cellular population. Molecular marker methods can be used for phylogenetic studies, characterizing genetic relationships among host cell varieties, identifying crosses or somatic hybrids, localizing chromosomal segments affecting monogenic traits, map-based cloning, and the study of quantitative inheritance. See, e.g., Clark, Ed., Plant Molecular Biology: A Laboratory Manual. Berlin, Springer Verlag, 1997. Chapter 7. For molecular marker methods, see generally, “The DNA Revolution” in: Paterson, A. H., Genome Mapping in Plants (Austin, Tex., Academic Press/R. G. Landis Company, 1996) pp. 7-21.

The particular method of genotyping in the present invention may employ any number of molecular marker analytic techniques such as, but not limited to, restriction fragment length polymorphisms (RFLPs) or next generation sequencing. RFLPs are the product of allelic differences between DNA restriction fragments resulting from nucleotide sequence variability. As is well known to those of skill in the art, RFLPs are typically detected by extraction of genomic DNA and digestion with a restriction enzyme. Generally, the resulting fragments are separated according to size and hybridized with a probe; single copy probes are preferred. Restriction fragments from homologous chromosomes are revealed.

Differences in fragment size among alleles represent an RFLP. Thus, the present invention further provides a means to follow segregation of a gene or nucleic acid of the present invention as well as chromosomal sequences genetically linked to these genes or nucleic acids using such techniques as RFLP analysis. Linked chromosomal sequences are within 50 centiMorgans (cM), often within 40 or 30 cM, preferably within 20 or 10 cM, more preferably within 5,3,2, or 1 cM of a gene of the present invention.

In the present invention, the nucleic acid probes employed for molecular marker mapping of nuclear genomes selectively hybridize, under selective hybridization conditions, to a gene encoding a polynucleotide of the present invention. In preferred embodiments, the probes are selected from polynucleotides of the present invention.

Typically, these probes are cDNA probes or restriction-enzyme treated (e.g., Pst I) genomic clones. The length of the probes is discussed in greater detail, supra, but are typically at least 15 bases in length, more preferably at least 20, 25, 30, 35, 40, or 50 bases in length. Generally, however, the probes are less than about 1 kilobase in length. Preferably, the probes are single copy probes that hybridize to a unique locus in a haploid chromosome complement. Some exemplary restriction enzymes employed in RFLP mapping are EcoRI, EcoRv, and Sstl. As used herein the term “restriction enzyme” includes reference to a composition that recognizes and, alone or in conjunction with another composition, cleaves at a specific nucleotide sequence. The method of detecting an RFLP comprises the steps of (a) digesting genomic DNA of a cell with a restriction enzyme; (b) hybridizing a nucleic acid probe, under selective hybridization conditions, to a sequence of a polynucleotide of the present of said genomic DNA; (c) detecting therefrom a RFLP.

Next generation sequencing can be performed by multiple methods, including, but not limited to, single-molecule real-time sequencing, ion semiconductor, pyrosequencing, sequencing by ligation, nanopore, or chain termination. Each next generation sequencing method has its own procedure known in the art. Prepping the DNA may be done through first isolating the DNA using techniques well known in the art. The isolated DNA may then be shortened to the appropriate length for sequencing such as by using sonication, enzymatic shearing. Libraries were then made by performing end repair, A-tailing, ligation, and amplification.

The resulting sequences are then assembled into consensus sequences. The consensus sequences across the samples may then be compared by aligning part or all of the genome to other portions or full genomes, and differences in the sequences may them be determined.

Other methods of differentiating polymorphic (allelic) variants of polynucleotides of the present invention can be had by utilizing molecular marker techniques well known to those of skill in the art including such techniques as: 1) single stranded conformation analysis (SSCA); 2) denaturing gradient gel electrophoresis (DGGE); 3) RNase protection assays; 4) allele-specific oligonucleotides (ASOs);5) the use of proteins which recognize nucleotide mismatches, such as the E. coli mutS protein; and 6) allele-specific PCR. Other approaches based on the detection of mismatches between the two complementary DNA strands include clamped denaturing gel electrophoresis (CDGE); heteroduplex analysis (HA); and chemical mismatch cleavage (CMC). Thus, the present invention further provides a method of genotyping comprising the steps of contacting, under stringent hybridization conditions, a sample suspected of comprising a polynucleotide of the present invention with a nucleic acid probe. Generally, the sample is a bacterial sample; preferably, a sample suspected of comprising an Enterococcus spp. polynucleotide of the present invention (e. g., gene, mRNA). The nucleic acid probe selectively hybridizes, under stringent conditions, to a subsequence of a polynucleotide of the present invention comprising a polymorphic marker. Selective hybridization of the nucleic acid probe to the polymorphic marker nucleic acid sequence yields a hybridization complex. Detection of the hybridization complex indicates the presence of that polymorphic marker in the sample. In preferred embodiments, the nucleic acid probe comprises a polynucleotide of the present invention.

### UTRs and Codon Preference

In general, translational efficiency has been found to be regulated by specific sequence elements in the 5′ non-coding or untranslated region (5′UTR) of the RNA. Positive sequence motifs include translational initiation consensus sequences (Kozak, Nucleic Acids Res. 15: 8125 (1987)) and the 7-methylguanosine cap structure (Drummond et al., Nucleic Acids Res. 13: 7375 (1985)). Negative elements include stable intramolecular 5′UTR stem-loop structures (Muesing et al., Cell 48: 691 (1987)) and AUG sequences or short open reading frames preceded by an appropriate AUG in the 5′UTR (Kozak, supra, Rao et al., Mol. and Cell. Biol. 8: 284 (1988)). Accordingly, the present invention provides 5′ and/or 3′ untranslated regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host such as to optimize the codon usage in a heterologous sequence for expression in bacteria. Codon usage in the coding regions of the polynucleotides of the present invention can be analyzed statistically using commercially available software packages such as “Codon Preference” available from the University of Wisconsin Genetics Computer Group (see Devereaux et al., Nucleic Acids Res. 12: 387-395 (1984)) or MacVector 4.1 (Eastman Kodak Co., New Haven, Conn.). Thus, the present invention provides a codon usage frequency characteristic of the coding region of at least one of the polynucleotides of the present invention. The number of polynucleotides that can be used to determine a codon usage frequency can be any integer from 1 to the number of polynucleotides of the present invention as provided herein. Optionally, the polynucleotides will be full-length sequences. An exemplary number of sequences for statistical analysis can be at least 1, 5, 10, 20, 50, or 100.

### Sequence Shuffling

The present invention provides methods for sequence shuffling using polynucleotides of the present invention, and compositions resulting therefrom. Sequence shuffling is described in PCT publication No. WO 97/20078. See also, Zhang, J.-H., et al. Proc. Natl. Acad. Sci. USA 94: 4504-4509 (1997). Generally, sequence shuffling provides a means for generating libraries of polynucleotides having a desired characteristic which can be selected or screened for. Libraries of recombinant polynucleotides are generated from a population of related sequence polynucleotides which comprise sequence regions which have substantial sequence identity and can be homologously recombined in vitro or in vivo. The population of sequence-recombined polynucleotides comprises a subpopulation of polynucleotides which possess desired or advantageous characteristics, and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property. In some embodiments, the selected characteristic will be a decreased Km and/or increased Kcat over the wild-type protein as provided herein. In other embodiments, a protein or polynucleotide generated from sequence shuffling will have a ligand binding affinity greater than the non-shuffled wild-type polynucleotide. The increase in such properties can be at least 110%, 120%, 130%, 140% or at least 150% of the wild-type value.

### Generic and Consensus Sequences

Polynucleotides and polypeptides of the present invention further include those having: (a) a generic sequence of at least two homologous polynucleotides or polypeptides, respectively, of the present invention; and, (b) a consensus sequence of at least three homologous polynucleotides or polypeptides, respectively, of the present invention. The generic sequence of the present invention comprises each species of polypeptide or polynucleotide embraced by the generic polypeptide or polynucleotide sequence, respectively. The individual species encompassed by a polynucleotide having an amino acid or nucleic acid consensus sequence can be used to generate antibodies or produce nucleic acid probes or primers to screen for homologs in other species, genera, families, orders, classes, phyla, or kingdoms.

Alternatively, a polynucleotide having a consensus sequence generated from orthologous genes can be used to identify or isolate orthologs of other taxa. Typically, a polynucleotide having a consensus sequence will be at least 25, 30, or 40 amino acids in length, or 20, 30, 40, 50, 100, or 150 nucleotides in length. As those of skill in the art are aware, a conservative amino acid substitution can be used for amino acids which differ amongst aligned sequence but are from the same conservative substitution group as discussed above. Optionally, no more than 1 or 2 conservative amino acids are substituted for each 10 amino acid length of consensus sequence.

Similar sequences used for generation of a consensus or generic sequence include any number and combination of allelic variants of the same gene, orthologous, or paralogous sequences as provided herein. Optionally, similar sequences used in generating a consensus or generic sequence are identified using the BLAST algorithm's smallest sum probability (P(N)). Various suppliers of sequence-analysis software are listed in chapter 7 of Current Protocols in Molecular Biology, F. M. Ausubel et al., Eds., Current Protocols, a joint venture between Greene Publishing Associates, Inc. and John Wiley & Sons, Inc. (Supplement 30).

A polynucleotide sequence is considered similar to a reference sequence if the smallest sum probability in a comparison of the test nucleic acid to the reference nucleic acid is less than about 0.1, more preferably less than about 0.01, or 0.001, and most preferably less than about 0.0001, or 0.00001. Similar polynucleotides can be aligned and a consensus or generic sequence generated using multiple sequence alignment software available from a number of commercial suppliers such as the Genetics Computer Group's (Madison, Wis.) PILEUP software, Vector NTI's (North Bethesda, Md.) ALIGNX, or Genecode's (Ann Arbor, Mich.) SEQUENCHER. Conveniently, default parameters of such software can be used to generate consensus or generic sequences.

### Stacking

In certain embodiments, the nucleic acid sequences of the embodiments can be stacked with any combination of polynucleotide sequences of interest in order to create host cells or organisms with a desired phenotype. This stacking may be accomplished by a combination of genes within the DNA construct, or by crossing a line containing the resistance genes of the invention (as transgenes or as an introgressed locus), with another line that comprises the combination in a sexually reproducing organism. For example, the polynucleotides of the embodiments may be stacked with any other polynucleotides of the embodiments, or with other genes. The combinations generated can also include multiple copies of any one of the polynucleotides of interest. The polynucleotides of the embodiments can also be stacked with any other gene or combination of genes to produce host cells or organisms with a variety of desired trait combinations including and not limited to traits desirable for the uptake of L-DOPA, such as catecholamine transporters such as, but not limited to, plasma membrane monoamine transporter (PMAT) and organic cation transporters (OCTs).

These stacked combinations can be created by any method including and not limited to cross breeding sexually reproductive organisms by any conventional or TOPCROSS® methodology, or genetic transformation or transfection. If the traits are stacked by genetically transforming or transfecting the host cell, the polynucleotide sequences of interest can be combined at any time and in any order. For example, a transgenic host cell comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis).

Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant.

By way of nonlimiting example, beneficial nucleic acids which may be stacked may include sequences encoding proteins which allow for transport proteins, such as but not limited to permeases like tyrosine or tyramine permease, or enzymes which may convert other substrates into L-DOPA or dopamine, such as hydroxylases like phenol 2-monoxygenase.

### Assays for Detecting Aromatic Decarboxylase

In the present invention, a sample of genetic material and/or protein is obtained from a cell colony, an animal, or a human. Samples can be obtained from blood, tissue, semen, etc. Generally, a cell colony or a fecal sample is used as the source, and the genetic material is RNA. A sufficient amount of cells are obtained to provide a sufficient amount for analysis. This amount will be known or readily determinable by those skilled in the art. The RNA and or protein is isolated from the sample by techniques known to those skilled in the art.

The term “semiquantitative PCR” refers to a kind of polymerase chain reaction (PCR), which can be carried out on tissue samples, on serum and plasma using primers specific to an aromatic decarboxylase without a probe, and the term “qPCR” or “QPCR” refers to quantitative PCR, which can be carried out on samples using aromatic decarboxylase specific primers and probes. In controlled reactions, the amount of product formed in a PCR reaction correlates with the amount of starting template (Sambrook, J., E Fritsch, E. and T Maniatis, Molecular Cloning: A Laboratory Manual 3^{rd} Cold Spring Harbor Laboratory Press: Cold Spring Harbor (2001)). In semiquantitative PCR, quantification of the PCR product can be carried out by stopping the PCR reaction when it is in log phase, before reagents become limiting. The PCR products are then electrophoresed in agarose or polyacrylamide gels, stained with ethidium bromide or a comparable DNA stain, such as Sybr Green, and the intensity of staining measured by densitometry.

In qPCR, the progression of a PCR reaction can be measured in real time using PCR machines such as the Applied Biosystems' Prism 7000 or the Roche LightCycler which measure product accumulation in real-time. Real-time PCR measures either the fluorescence of DNA intercalating reporter dyes such as Sybr Green into the synthesized PCR product, or the fluorescence released by a reporter dye, such as, but not limited to cy3, cy5, FAM, SYBR Green, HEX™, JOE, TAMRA, Tye™ 563, TEX 615™, Tye™ 665, VIC, and/or LC Red 640, when cleaved from a quencher molecule, where the quencher molecule prevents the reporter dye from being detectable; the reporter and quencher molecules are incorporated into an oligonucleotide probe which hybridizes to the target DNA molecule following DNA strand extension from the primer oligonucleotides. The oligonucleotide probe is displaced and degraded by the enzymatic action of the DNA polymerase in the next PCR cycle, releasing the reporter from the quencher molecule, allowing the reporter dye to be detectable. In one variation, known as ScorpionTM, the probe is covalently linked to the primer.

Reverse Transcription PCR (RT-PCR) can be used to compare RNA levels in different sample populations to characterize patterns of expression, to discriminate between closely related RNAs, and to analyze RNA structure.

For RT-PCR, the first step is the isolation of RNA from a target sample. The starting material is typically total RNA isolated from an animal. The first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukaemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan qPCR typically utilizes the 5′ nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction.

A third oligonucleotide, or probe, used in real-time qPCR is designed to detect nucleotide sequence located between the two PCR primers. The probe is non-extendible by Taq DNA polymerase enzyme and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

RT-PCR can be performed using commercially available equipment, such as, for example, ABI PRISM 7700 Sequence Detection System (Perkin-Elmer-Applied Biosystems, Foster. City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 Sequence Detection System. The system consists of a thermocycler, laser, charge-coupled device (CCD), camera, and computer. The system amplifies samples in a multi-well format on a thermocycler. During amplification, laser-induced fluorescent signal is collected in real-time through fibre optics cables for each well and detected at the CCD. The system includes software for running the instrument and for analyzing the data.

5′ nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle.

Real-Time Quantitative PCR (qRT-PCR) is a more recent variation of the RT-PCR technique is the real time quantitative PCR, which measures PCR product accumulation through a dual-labeled fluorogenic probe (i.e., TaqMan probe). Real time PCR is compatible both with quantitative competitive PCR and with quantitative comparative PCR. The former uses an internal competitor for each target sequence for normalization, while the latter uses a normalization gene contained within the sample, or a housekeeping gene for RT-PCR. Further details are provided, e.g., by Held et al., Genome Research 6: 986-994 (1996). If introns are present, PCR primers are designed to flank intron sequences present in the gene to be amplified. In this embodiment, the first step in the primer/probe design is the delineation of intron sequences within the genes. This can be done by publicly available software, such as the DNA BLAT software developed by Kent, W. J., Genome Res. 12 (4): 656-64 (2002), or by the BLAST software including its variations. Subsequent steps follow well established methods of PCR primer and probe design.

In order to avoid non-specific signals, it is useful to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the VIMNV for general users and for biologist programmers in: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′ end sequence. In general, optimal PCR primers are generally 17-30 bases in length, and contain about 20-80% G+C bases, such as, for example, about 50-60% G+C bases. Melting temperatures between 50 and 80° C., e.g., about 50 to 70° C., are typically preferred. For further guidelines for PCR primer and probe design see, e.g., Dieffenbach, C. W. et al., General Concepts for PCR Primer Design in: PCR Primer, A Laboratory Manual, Cold Spring Harbor Laboratory Press, New York, 1995, pp. 133-155; Innis and Gelfand, Optimization of PCRs in: PCR Protocols, A Guide to Methods and Applications, CRC Press, London, 1994, pp. 5-11; and Plasterer, T. N. Primerselect: Primer and probe design. Methods Mol. Biol. 70: 520-527 (1997), the entire disclosures of which are hereby expressly incorporated by reference.

Enzyme-linked immunosorbent assay (ELISA) and sandwich ELISA, are immunoassays that are advantageously used in the methods disclosed herein. In a (direct) ELISA, for example, an unknown amount of antigen (i.e., aromatic decarboxylase nucleotides, aromatic decarboxylase peptides, or aromatic decarboxylase protein) is affixed to a substrate, and then a specific antibody is applied over the surface so that it can bind to the antigen. This antibody is conjugated to a reporter, such as, but not limited to, alkaline phosphatase, peroxidase, β-galactosidase, Atto 425, Atto 488, Cy2, DyLight 405, DyLight 488, Atto 432 Atto 550, Cy3, Cy5, DyLight 549, TEX 615™, Allophycocyanin, Atto 647, DyLight 649, Atto 655, Cy5.5, Dylight 680, and/or DyLight 800, and, in the case of an enzyme being the conjugate, an enzyme, and in the final step a substance is added so that the enzyme can convert to some detectable signal, most commonly a color change in a chemical substrate. In a sandwich ELISA a capture antibody that can bind to the antigen is affixed to the substrate. The other steps are equivalent to the ELISA. The detectable signal can be detected by a number of commercially available systems, such as, but not limited to, the Bio-Rad iMark Readers, BioTek Synergy Readers (Winooski, Vt.), or various BMG LABTECH star Readers (Cary, N.C.).

In an Enzyme Immuno Assay (EIA), which is similar to the sandwich ELISA, streptavidin is affixed to a surface and then the capture antibody is biotinylated, otherwise the other steps are performed equivalently as the ELISA. The EIA immunoassay is advantageously used in the methods disclosed herein. The detectable signal can be detected by a number of commercially available systems, such as, but not limited to, the Bio-Rad iMark Readers, BioTek Synergy Readers (Winooski, Vt.), or various BMG LABTECH star Readers (Cary, N.C.).

In a blotting assay, such as Western Blot, the sample is separated out in an appropriate gel and then transferred to a substrate, such as, but not limited to, nitrocellulose or PVDF. The membrane is then blocked to prevent nonspecific protein binding, followed by incubating the blot with antigen specific antibodies. The antigen specific antibodies can be conjugated with a reporter, such as, but not limited to, alkaline phosphatase, peroxidase, β-galactosidase, Atto 425, Atto 488, Cy2, DyLight 405, DyLight 488, Atto 432 Atto 550, Cy3, Cy5, DyLight 549, TEX 615™, Allophycocyanin, Atto 647, DyLight 649, Atto 655, Cy5.5, Dylight 680, and/or DyLight 800, or can be further bound by a secondary antibody conjugated to a reporter that can bind to the antigen specific antibody. Like in an ELISA, the blot can either be read directly if a reporter dye is conjugated or if an enzyme is conjugated, the appropriate substance is added to the blot in order to create a detectable signal. The signal can them be detected by a number of commercially available systems, such as, but not limited to, the Bio-Rad ChemiDoc (Hercules, Calif.) or a Typhoon Biomolecular Imager (GE Healthcare Life Sciences, Malborough, Mass.).

One of ordinary skill in the art will also understand that aromatic decarboxylases can also be part of high throughput assays. These high throughput assays may not be specifically designed to assay only aromatic decarboxylases, but rather the expression of multiple RNAs, cDNAs, or proteins at once. Examples of such high throughput assays include, but are not limited to, spotted oligonucleotide microarrays, printed oligonucleotide microarrays, protein microarrays, single molecule real-time sequencing, ion semiconductor sequencing, pyrosequencing, sequencing by synthesis, sequencing by ligation, nanopore sequencing, chain termination sequencing, tunneling currents DNA sequencing, sequencing by hybridization, sequencing with mass spectrometry, microscopy-based DNA sequencing, RNA polymerase (RNAP) sequencing, and/or in vitro virus high throughput sequencing.

Other detection methods, such as, but not limited to, strand displacement circuits, are also contemplated by the invention.

Kits Based on the discoveries of this invention, several types of kits can be envisioned and produced. One embodiment of the kit comprises the primers, reagents, and instructions for assaying the expression of an aromatic decarboxylase using any of the described PCR methods. These kits would comprise: oligonucleotide primers and/or probes labeled with a reporter dye and quencher which specifically bind to an aromatic decarboxylase to measure the expression of an aromatic decarboxylase; PCR reagents; and instructions for use, either within the kit or available online. Other kits may analysis aromatic decarboxylase protein and comprise: one or more antibody(ies) to an aromatic decarboxylase, which may have been conjugated to a reporter as described herein; an optional secondary antibody that binds to an aromatic decarboxylase specific antibody and is has been conjugated to a reporter as described herein; reagents appropriate for the kind of protein capture used, such as ELISA, EIA, or blotting reagents; and instructions for use. The protein kits may optionally further comprise of the substance which corresponds to the conjugated reporter. Alternatively, the kits can further comprise a substrate, such as a glass slide, a multiwell plate, or nitrocellulose paper, which the capture molecule, such as an oligonucleotide or antibody, may be bound to. Optionally, the kit can further comprise any other regent, such as, but not limited to, hybridizing buffer and label, for identification of aromatic decarboxylases in biological samples, using a specific probe. Further kits my assay aromatic decarboxylases in addition to one or more non- aromatic decarboxylases RNA, cDNA, and/or proteins, such as to, but not limited to, housekeeping genes, and would further comprise of capture molecules for the one or more non-aromatic decarboxylases molecules.

### Systems for Assaying

A “system” as used herein refers to a sample, a kit, and a device to detect the signal produced by the kit. The sample may be drawn blood, a tissue biopsy, lung lavage, sperm, the kit may be any of the kits contemplated herein, and the device may include any of the variety of available for the various assay methods described herein. For example, for detecting the bands resulting from semiquantitative PCR, an image may be taken using a camera, and the bands quantified using software such as, but not limited to, ImageJ (NIH, Bethesda, Md.). ImageJ may also be used to quantify the bands created during blots, such as, but not limited to, Western blotting.

Florescent reporters can be read on systems such as, but not limited to, ABI PRISM 7700 Sequence Detection System or Lightcycler for PCR based assays, or on a Bio-Rad ChemiDoc or a Typhoon Biomolecule Imager for blots. ELISA and EIA can be read on devices such as, but not limited to, the Bio-Rad iMark Readers, BioTek Synergy Readers, or various BMG LABTECH star Readers.

### Production of Dopamine

In an embodiment, dopamine is produced by a cell comprising of an aromatic decarboxylase which is capable of decarboxylating L-DOPA into dopamine. In a further embodiment, the cell has been transfected or transformed with an aromatic decarboxylase to improve its dopamine function when compared to a cell of the same type without the aromatic decarboxylase. By way of a nonlimiting example, strains of bacteria may be screened for the presence of aromatic decarboxylase and then selected for a specific genotype. Bacteria with an aromatic decarboxylase may then be cultured in a media, such as media simulating the gastrointestinal tract to encourage growth, which may be supplemented with L-DOPA. After about 2, about 3, about 4, about 6, about 8, about 10, about 12, about 16, about 24 hours, about 48, about 72 hours, about 1 week, about 2 weeks, or for as long as the culture or retained dopamine remains viable, the dopamine may then be purified from the media. In a preferred embodiment, dopamine is purified from about 24 to 72 hours after culture. In a preferred embodiment, the cell is an Enterococcus spp.

In a further embodiment, the transfected or transformed cell may be either a prokaryote or eukaryote cell. For example, the cell may be bacterial, fungus, insect, plant, animal, or human. By way of a nonlimiting example, a cell with good growth in a desirable media may be transformed or transfected such that said cell has increased or is conferred dopamine production.

In another embodiment, a transfected or transformed cell with aromatic decarboxylase may be stacked with a catecholamine transporter. This may allow a cell increased dopamine production due to an increased ability to both uptake and then process L-DOPA into dopamine.

In an embodiment, the L-DOPA is provided as a food source or a purified compound. By way of nonlimiting example, the L-DOPA may be provided as a Mucuna puriens powder or Vicia faba green pods, which are high in L-DOPA.

As used herein, “Mucuna” is used interchangeably with Mucuna puriens, and as such, “Mucuna powder” may also be used interchangeably with “Mucuna puriens powder.”

Cells may be cultured in a bioreactor. The term “bioreactor” refers to any manufactured or engineered device or system that supports a biologically active environment. By way of nonlimiting example, a bioreactor may be a single-use or multi-use flask, roller bottle, tank, vessel, or other container which may support the growth of a cell culture. The bioreactor may comprise of an agitator, baffle, sparger, and/or a jacket. The bioreactor may be an open or closed system. An open system may allow the culture to be fed a continuous or intermittent supply of L-DOPA. The cells may be fixed to a substrate within the bioreactor or adhered to the inner surface of the bioreactor.

In another embodiment, cells are grown inside the gut of an animal and/or human.

Extraction of the dopamine from the media may be done by any means, such as, but not limited to, filtration, pressure, or affinity bead extraction.

### Inhibition of Dopamine Production

In an embodiment, dopamine produced by a cell comprising an aromatic decarboxylase which is capable of decarboxylating L-DOPA into dopamine is inhibited. In a further embodiment, the cell comprising an aromatic decarboxylase which may produce dopamine produces less dopamine when compared to a cell of the same type without the inhibitor. Bacteria with an aromatic decarboxylase may be cultured in a media, such as media simulating the gastrointestinal tract to encourage growth, which may be supplemented with an inhibitor. The inhibitor may be a genetic inhibitor, such as a plasmid comprising siRNA or a nuclease which may result in the knock out of expression of the aromatic decarboxylase which is capable of decarboxylating L-DOPA into dopamine; or a small molecule, such as, but not limited to carbidopa, deoxypyridoxine phosphate, a fluoromethyl amino acid, and/or or side group substitutions as well as analogs or enantiomers thereof. Examples of fluoromethyl amino acids include, but are not limited to, α-fluoromethyltyrosine, α-fluoromethyl(3,4-dihydroxyphenyl)alanine, α-aminooxy-β-phenylpropiomate, as well as other halogenated equivalents or side group substitutions as well as analogs or enantiomers thereof. In a preferred embodiment, the cell is an Enterococcus spp. The inhibitor may also be administered to the gastrointestinal tract of a subject to inhibit the production of dopamine by gut bacteria.

The inhibitor may be administered to gut from about 0.0001 mM to about 50 mM, about 0.01 mM to about 15 mM, from about 0.5 mM to about 5 mM, or from about 0.75 mM to about 1.5 mM.

In a further embodiment, the cell may be either a prokaryote or eukaryote cell. For example, the cell may be bacterial, fungus, insect, plant, animal, or human.

Cells may be cultured in a bioreactor. The term “bioreactor” refers to any manufactured or engineered device or system that supports a biologically active environment. By way of nonlimiting example, a bioreactor may be a single-use or multi-use flask, roller bottle, tank, vessel, or other container which may support the growth of a cell culture. The bioreactor may comprise of an agitator, baffle, sparger, and/or a jacket. The bioreactor may be an open or closed system. An open system may allow the culture to be fed a continuous or intermittent supply of L-DOPA. The cells may be fixed to a substrate within the bioreactor or adhered to the inner surface of the bioreactor.

In another embodiment, cells are grown inside the gut of an animal and/or human.

In another embodiment, the inhibition of dopamine production in a cell comprising an aromatic decarboxylase which is capable of decarboxylating L-DOPA into dopamine inhibits the production of dopamine derived compounds in the cell or in cells in coculture or present in the same environment.

All publications, patent applications, issued patents, and other documents referred to in this specification are indicative of the level of ordinary skill in the art to which this invention pertains and are herein incorporated by reference to the same extent as if each individual publication or patent application was specifically and individually indicated as incorporated by reference. Definitions that are contained in text incorporated by reference are excluded to the extent that they contradict definitions in this disclosure.

The present invention is further illustrated by the following examples, which should not be considered as limiting in any way.

### EXAMPLES

### Example 1

In order to first establish that E. faecium strains are capable of producing neurochemicals, the dopamine production of probiotic strains were evaluated. The evaluation of a commercially available probiotic formulation Probios® containing four probiotic species, including E. faecium, was conducted to determine the ability to produce the neurochemical dopamine. The probiotic strains were grown anaerobically for twenty-four hours on TSA agar with 5% ovine blood. Following plate growth, colonies were suspended in peptone water to make standardized suspensions for inoculation. Each suspension was adjusted such that the material to be inoculated had an OD₆₀₀ measurement of 0.200 (+/−0.005). The probiotic strains inoculated a specialized medium which closely mimics the environment of the gastrointestinal tract, simulated Small Intestinal Media (sSIM), designed to reflect physiological conditions in the gastrointestinal tract. 4.8 mL of sSIM was spiked with 100 of a 0.05 M L-DOPA solution. L-DOPA solution was prepared by weighing dry L-DOPA purchased from Sigma and dissolving it in 0.1M HCl. For a sample with a total volume of 5 mL, 4.8 mL of sSIM was mixed with 100 μL of L-DOPA spiking solution and 100 μL of bacterial suspension. This yielded a final L-DOPA concentration of 1 mM. Inoculated samples were grown at 37° C. anaerobically while being subjected to low speed (100 rpm) magnetic stir bar mixing. All conditions were run in triplicate.

The L-DOPA concentration was set to be high enough to be non-limiting in order to better resolve L-DOPA usage differences between various strains of Enterococci. At lower concentrations of L-DOPA, it is possible for all available L-DOPA to be consumed without reliably distinguishing the strains capable of greater production. Further, this concentration falls into a meaningful physiological range. Every milliliter of sSIM derives from the digestion of 125 mg of raw undigested dry material. As each mL of sSIM is spiked with 197 μg of L-DOPA in addition to the 10 μg/mL baseline provided by the sSIM, the total L-DOPA content would be roughly equivalent to a food consisting of 0.17% L-DOPA by mass. Naturally L-DOPA rich foods, like the green pods of broad/fava beans (Vicia faba), can reach concentrations as high as 6.75%. Similar L-DOPA concentrations might also be achieved by imbibing a tablet containing 100 mg L-DOPA (common prescription strength) along with a small 60-gram snack.

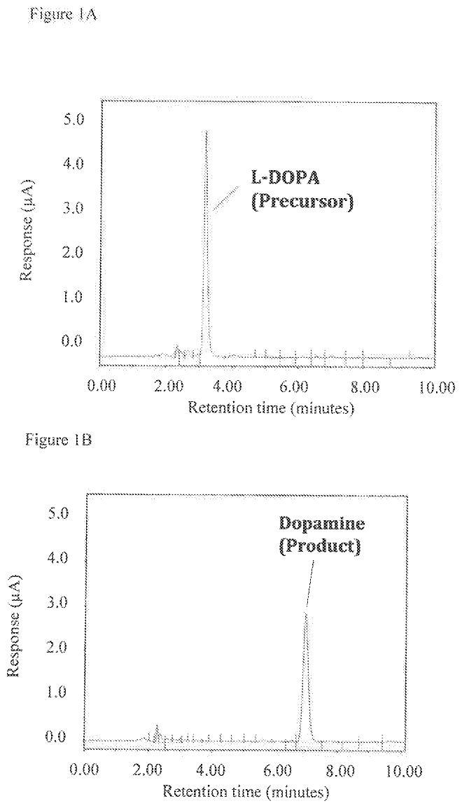

The results are shown in FIG. 1 where the Probios® product produced dopamine in the medium. The two HPLC chromatograms compare a control to the medium inoculated with a probiotic. The HPLC separates the individual components apart and the area under each peak determines the concentration of each metabolite. The peak for dopamine is much larger and the L-DOPA peak greatly decreases in the presence of the probiotic. Further characterization revealed that only the E. faecium component in the product was producing dopamine (data not shown). This shows that the E. faecium strains of Probios® are capable of converting L-DOPA into dopamine in the sSIM media.

### Example 2

In order to evaluate additional strains, a similar evaluation was completed on additional commercially available probiotics, Fortiflora which contains only E. faecium, and E. faecium strains isolated from the environment (see Table 1). The probiotic strains of E. faecium were isolated from Probios and Fortiflora using standard microbiological techniques and identification by MALDI. All isolates identified by MALDI had highly reliable identification scores of >2.4.

The probiotic strains were grown and plated on medium as described in Example 1. The evaluated strains and sources thereof, along with the results of production of the dopamine in the medium are shown in Table 1. The conversion efficiency was calculated as follows:

$$= \frac{{[ L - DOPA ]}_{i} - {[ L - DOPA ]}_{f}}{{[ L - DOPA ]}_{i}} \times \frac{{[ Dopamine ]}_{f} - {[ Dopamine ]}_{i}}{{[ L - DOPA ]}_{i} - {[ L - DOPA ]}_{f}} \times 100 \\ = \frac{{[ Dopamine ]}_{f} - {[ Dopamine ]}_{i}}{{[ L - DOPA ]}_{i}} \times 100$$

TABLE 1

| | | Avg. | Avg. | L-DOPA | Dopamine | Conversion |
|---|---|---|---|---|---|---|
| | | L-DOPA | Dopamine | Consumed | Produced | Efficiency |
| Strain | Source | (μg/mL) | (μg/mL) | (μg) | (μg) | (%) |
| CONTROL | | 179.10 ± 0.99 | 2.00 ± 0.01 | 0.00 | 0.00 | |
| ML1081 | Fortiflora | 32.93 ± 0.37 | 89.75 ± 1.71 | 146.18 | 87.75 | 63.1 |
| ML1082 | Probios | 2.61 ± 0.05 | 135.73 ± 2.65 | 176.49 | 133.73 | 96.1 |
| ML1085 | Caine | 52.34 ± 2.55 | 79.88 ± 1.31 | 126.76 | 77.88 | 56.0 |
| | urine | | | | | |
| ML1086 | Canine | 2.86 ± 0.31 | 108.00 ± 2.18 | 176.24 | 106.00 | 76.2 |
| | incision | | | | | |
| ML1087 | Feline | 167.43 ± 2.64 | 11.68 ± 0.70 | 11.68 | 9.68 | 7.0 |
| | urine | | | | | |
| ML1088 | Avian | 114.95 ± 4.61 | 38.89 ± 2.98 | 64.16 | 36.88 | 26.5 |
| | yolk sac | | | | | |
| ML1089 | Canine | 17.83 ± 2.90 | 106.45 ± 3.69 | 161.28 | 104.45 | 75.1 |
| | bile | | | | | |

UHPLC was used in order to determine the amount of the various catecholamines. Dopamine, L-DOPA and other metabolites were isocratically separated by a reversed-phase column at a flow rate of 0.6 ml min-1 using a Dionex Ultimate 3000 HPLC system (pump ISO-3100SD, Thermo Scientific, Bannockburn, Ill.) equipped with a refrigerated automatic sampler (model WPS-3000TSL). The electrochemical detection system included a CoulArray model 5600A coupled with an analytical cell (microdialysis cell 5014B) and a guard cell (model 5020). Data acquisition and analysis were performed using Chromeleon 7 and ESA CoulArray 3.10 HPLC Software.

To evaluate the efficiency of the ultra-high-performance liquid chromatography with electrochemical detection (UHPLC-ECD) system to extract dopamine from the media the amount of predicted dopamine return was compared to the actual dopamine return. First, to evaluate a control sample of sSIM, freshly prepared stock solution was made by dissolving 47 mg of dopamine hydrochloride (FW: 189.64) into 5 mL of HPLC grade water for a concentration of 0.05 M. One hundred μL of the stock solution was added to 4.9 mL of sSIM medium for a concentration of 0.001 M in a total volume of 5.0 mL. The mass of the dopamine (FW 153.18) component added would be expected to be 766 μg. Measurements of unsupplemented sSIM indicate only very trace quantities of dopamine (<2 μg/mL). For the purposes of this evaluation, 5 mL of sSIM should have 776 μg.

This estimate was then compared to the amount of dopamine returned by the UHPLC-ECD system. The 5 mL spiked sample was acidified by the addition of 504, of 10 N HCl and centrifuged at 3000 × g at 4° C. for 15 minutes to remove insoluble fiber, denatured proteins, and other precipitates. The supernatant was processed for UHPLC-ECD by passage through a 3 kDa molecular weight cut off filter. The UHPLC-ECD determination of dopamine concentration in the supernatant was 122 mcg/mL. To determine the total volume of supernatant, the volume of the dry pellet (0.275 grams) was subtracted from the total volume of 5 mL medium (4.691 grams) to yield the supernatant mass (4.416 grams). The density of the supernatant was determined to be 1.02 grams/mL. Thus, a final volume of 4.33 mL of supernatant was present in a typical 5 mL sSIM sample. 122 mcg dopamine/mL×4.33 mL yields the total dopamine mass of 528 mcg (recovery of 68.1%.) The discrepancy can likely be attributed to losses from the molecular exclusion filter as well as residual dopamine associated with material forming the pellet.

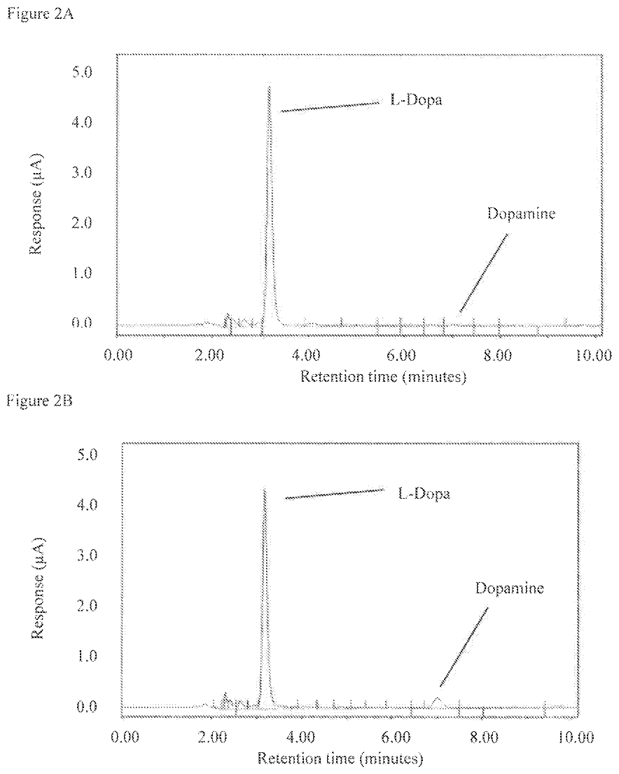

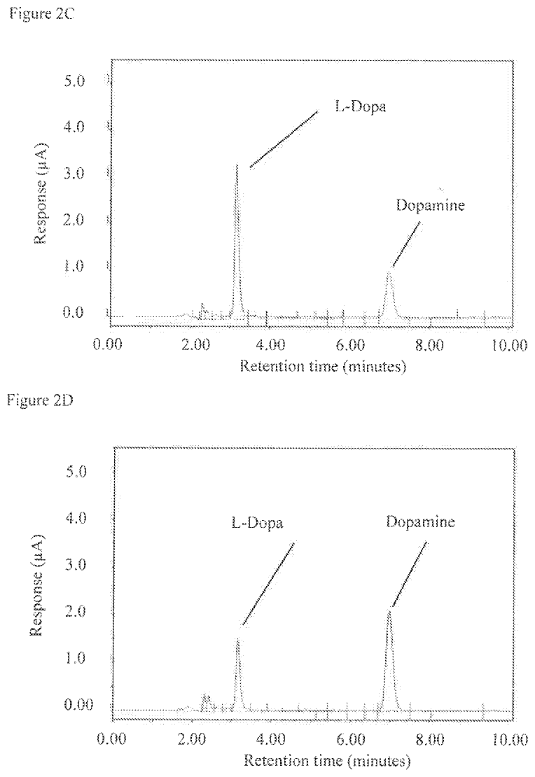

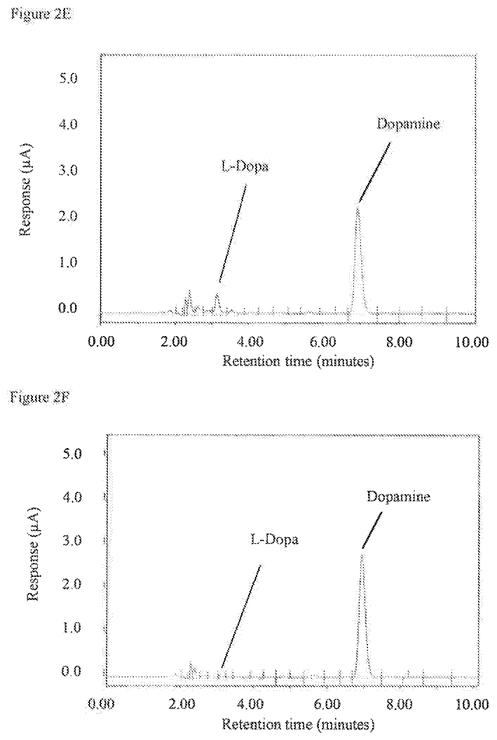

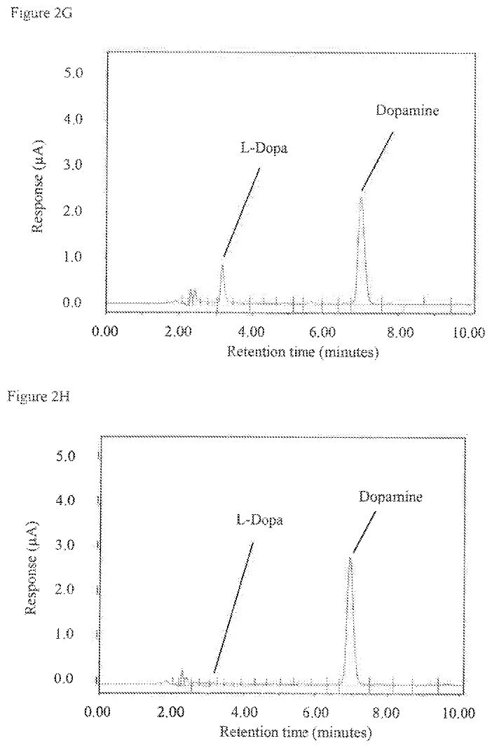

In addition to dopamine, L-DOPA recovery was also assayed using the same approach. Briefly, 197 μg L-DOPA/mL sSIM (227 μg L-DOPA/mL supernatant) is spiked in. Baseline sSIM supernatant contains about 10 μg/mL. For 4.33 mL of supernatant, a total L-DOPA mass of 983 μg is expected. Samples were determined by HPLC to have 180 μg L-DOPA/mL supernatant; therefore, a total of 779 μg was recovered yielding a total recovery of 79.2%. The results are shown in FIGS. 2A-2H where the strains of E. faecium grown in sSIM supplemented with 1.0×10⁻³ M L-DOPA are depicted. Both dopamine production and L-DOPA utilization efficacy were evaluated along with population differences between strains.

The results show the probiotic strain isolated from Probios® consistently demonstrated the greatest level of production at over 133 μg/mL. This production was more than 26% greater than the next highest producer, a clinical strain designated ML1086. Both of these strains demonstrated comparable levels of population growth and the consumption of L-DOPA appeared to be exhaustive in both of these samples with less than 2% of the starting L-DOPA remaining in both samples. Differences in the final dopamine concentrations of these samples appear to arise from differing efficiencies in the conversion of L-DOPA to dopamine. ML1082 demonstrated a conversion efficiency of 96%, 20% higher than the efficiency of ML1086.

The probiotic strain isolated from Probios® consistently demonstrated the greatest level of dopamine production (133 μg /mL, Conversion Efficiency (C. E.) 96%). Among the other samples, there was a high level of variation in the capacity to produce dopamine and in the ability to reproduce within the gastrointestinal-like contents of the sSIM. The strain ML1088 demonstrated one of the highest observed levels of population growth but was a relatively poor producer of dopamine (37 pg/mL, C. E. 27%). ML1087 produced the lowest amount of dopamine (10 μg/mL, C. E. 7%), likely due to poor growth in the sSIM (5.45×10⁷ CFU/mL). All other strains were able to achieve growths on the order of 1.0x10⁸ CFU/mL with prolific strains like ML1089 achieving population over a full order of magnitude greater than ML1087. This data confirms the capacity to produce dopamine from L-DOPA may be a common trait among members of the Enterococcus spp. as each tested strain demonstrated some capacity to generate dopamine.

However, the conversion efficiency of L-DOPA to dopamine varies greatly among individual E. faecium isolates (FIGS. 2A-2H). Thus, not all strains of E. faecium would be expected to be equally suitable for roles in the production of dopamine, such as in industrial applications. Notably, the results described herein demonstrate an ability of all the tested E. faecium strains to produce some amount of dopamine given the same input of L-DOPA, but some are better suited for production on larger scale than others.

### Example 3

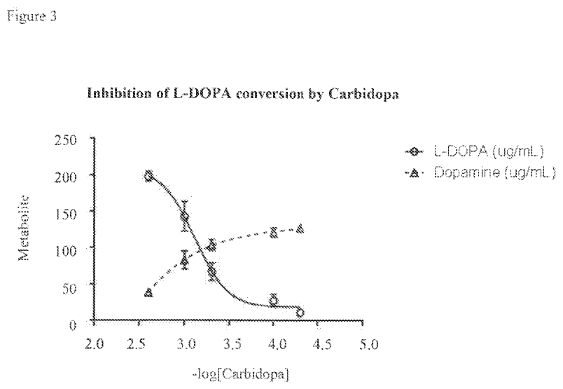

To further verify the strains were producing dopamine from L-DOPA, Carbidopa, an inhibitor of dopamine production through L-DOPA decarboxylation, was added to sSIM. The dopamine production of our most efficient strain, ML1082, demonstrated dose-dependent inhibition in the presence of the L-DOPA decarboxylase inhibitor Carbidopa (FIG. 3). A L-DOPA rich diet or a commonly prescribed 100 mg dose of L-DOPA would yield a concentration of about 1 mM in sSIM. It follows then that a tablet that has 1:10 Carbidopa:L-DOPA would likely result in a Carbidopa concentration of approximately 1/10th that on the order of 1.0×10⁻⁴M (-log [Carbidopa] =4).

FIG. 3 shows the concentration of carbidopa decreases (left to right on this reverse logarithmic plot). A functional approximation of the ICso for dopamine was calculated to be 1.53E⁻³. The IC₅₀ was similar to the L-DOPA concentration of the broth, 1.26 mM, a finding that may indicate a competitive inhibitory mechanism for the bacterial enzyme.

To further show that inhibition of the aromatic decarboxylase could affect downstream production of the formation of other compounds, E. faecium was cocultured with E. coli. E. coli is capable of producing large quantities of the neurotoxin salsolinol from dopamine produced in the gut. Therefore, to further show that the inhibition of dopamine production was occurring, as well as to test if inhibitors may prevent the buildup of unwanted products, E. coli production of salsolinol was measured.

Enterococci faecium was grown in co-culture with Escherichia coli in MRS supplemented with L-DOPA. Active cultures of respective organisms were suspended to achieve an OD of 0.2. To inoculate, 2004, of each organism suspension was introduced into 10 mL of MRS and samples were allowed to incubate aerobically at 37° C. for 24 hours. Dopamine was not supplied directly. Rather, the dopamine precursor L-DOPA was supplemented, and any dopamine produced resulted from the action of E. faecium. Subsequently, salsolinol was produced by activities derived from Escherichia coli. In order to examine whether inhibition of the bacterial decarboxylase enzyme could also decrease the accumulation of the neurotoxin salsolinol, the inhibitors deoxypyridoxine phosphate and carbidopa were tested. For the Carbidopa treated samples, 2004, of a 100 mM solution of carbidopa was added to inoculated broth solution to reach a carbidopa concentration of approximately 2 mM. Similarly, 2004, of a deoxypyridoxine phosphate containing preparation (see below) was used to prepare deoxypyridoxine containing samples.

Synthesis of deoxypyridoxine phosphate:

Deoxypridoxine phosphate was produced as in 1947, Beiler and Martin (1947, Inhibition of the action of tyrosine decarboxylase by phosphorylated desoxypyridoxine. J Biol Chem 169(2): 345-347, herein incorporated by reference in its entirety). Briefly, phosphorus V oxychloride was prepared by slowly adding 69 grams (44 mL) of phosphorus trichloride to 21 grams of potassium chlorate in a round bottom flask connected with a condenser and receiver. A vigorous reaction ensued in which phosphorous V oxychloride boiled off and was collected by the receiver. Collected phosphorus V oxychloride was further purified by re-distillation and collection of fractions near 110° C.

Next, 10 grams of sodium hydroxide was dissolved into 10 grams of water to create a 1:1 saturated solution of sodium hydroxide (solubility 1000 g/L at 25° C.). 88 mg of deoxypyridoxine was dissolved in 8.8 mL of water and 8004, of saturated 1:1 sodium hydroxide was added. The solution was chilled in an ice bath before 4004, of phosphorus V oxychloride and an additional 1.6 mL of saturated sodium hydroxide was added. The mixture solidified and was heated in a water bath at 70° C. to re-dissolve the precipitate. Four more cycles of this process of phosphorus V oxychloride addition were performed. Following the final addition, the material was transferred to a 50 mL conical tube and centrifuged at 5° C. to separate precipitate crystals from the deoxypyridoxine containing supernatant. Following precipitation, the supernatant was transferred to a fresh tube and placed at −20° C. to ensure no further crystallization occurred. 3 mL of supernatant was recovered. This was diluted to 12 mL and brought to a pH of 6.0 with the addition of 10N hydrochloric acid. 2004, of this solution was used to treat 10 mL of MRS for biological experiments.

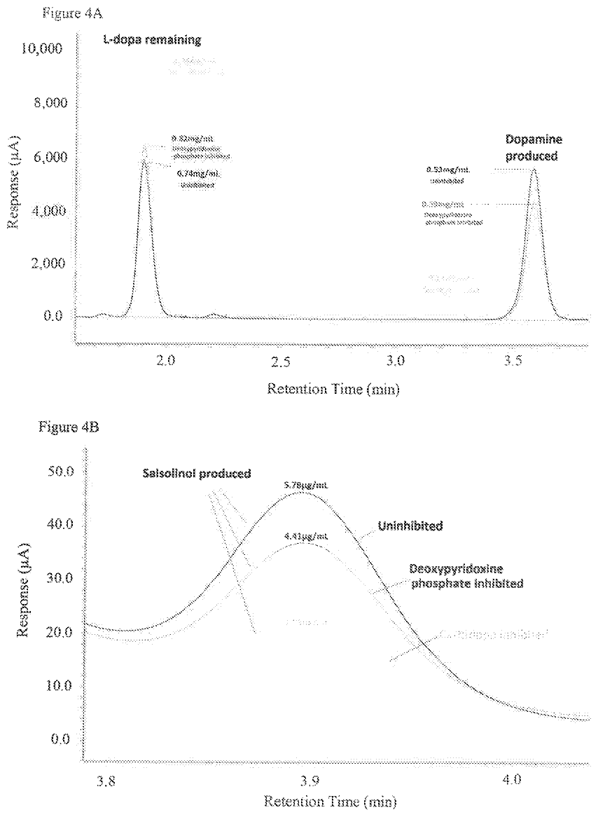

The inclusion of the inhibitors significantly impacts the generation of salsolinol (FIGS. 4A and 4B). Additionally, higher levels of L-DOPA remain when the inhibitors are applied which is of great clinical significance as more L-DOPA is available to cross the blood brain barrier. Taken together with the data above about inhibition by carbidopa in sSIM, this shows that not only are the cells producing dopamine through an aromatic decarboxylase, but the inhibitors may be used to help fine tune the production of dopamine in order to prevent over production and to prevent the production of neurotoxins, such as salsolinol, in a subject.

### Example 4

A medium like sSIM, which mimics the environment of the gastrointestinal tract, is relatively expensive to produce and complicates the extraction of valuable neurochemicals. For this reason, various E. faecium strains were tested in commercially available media spiked with L-DOPA. In order to provide a maximum amount of L-DOPA that could be consumed in 24-hours without waste, one of the most productive strains of E. faecium, ML1082, was provided a great excess of L-DOPA in the media, amounting to 20 mM, plus what is available in the media. The total amount of L-DOPA remaining after 24 hours was determined as above using UHPLC-ECD. About half of the available 20 mM L-DOPA was utilized (data not shown), indicating that strain ML1082 was able to consume around 10 mM of L-DOPA without waste.

Six different commercially available media were assayed for bacterial production of dopamine: Luria Broth, MRS, Tryptic Soy Broth, BHI, Nutrient Broth, and peptone water. L-DOPA was prepared by using a minimal about of acid to dissolve all the L-DOPA. L-DOPA was dissolved into HPLC grade water in the amount of 1.97 grams of L-DOPA into 7.5 mL HPLC grade water with the addition of 1.25 mL of 10 N HCl to yield a total volume of 10 mL. 100 μL of this L-DOPA solution was added to a total volume of 10 mL of each of the media tested to yield the target 10 mM.

Five strains of Enterococcus faecium, ML1085, ML1086, ML1087, ML1088, and ML1089, were grown in triplicate in the commercially available media with 10 mL L-DOPA. To inoculate, strains were grown aerobically, at 37° C., overnight on TSA agar plates containing 5% ovine blood. Cells were harvested and suspended in peptone water to achieve standard inoculation densities of 0.2 OD₆₀₀. 1004, of each respective suspension was used to inoculate 10 mL of media. Uninoculated tubes of the respective supplemented media were used as controls. Tubes were grown without agitation for 24 hours, aerobically at 37° C. At 24 hours, broth cultures were acidified with the addition of 1004, of 10N HCl. Acidified cultures were centrifuged at 3000xG, for 15 minutes at 4° C. to remove cells and denatured proteins. The supernatant was transferred to 3 kDa molecular weight cut off filter tubes and filtered by centrifugal force provided at 4300×G, at 4° C. Aliquots of respective samples were diluted 1000× into mobile phase and tested by UHPLC-ECD. Remaining sample partitions were stored at −80° C.

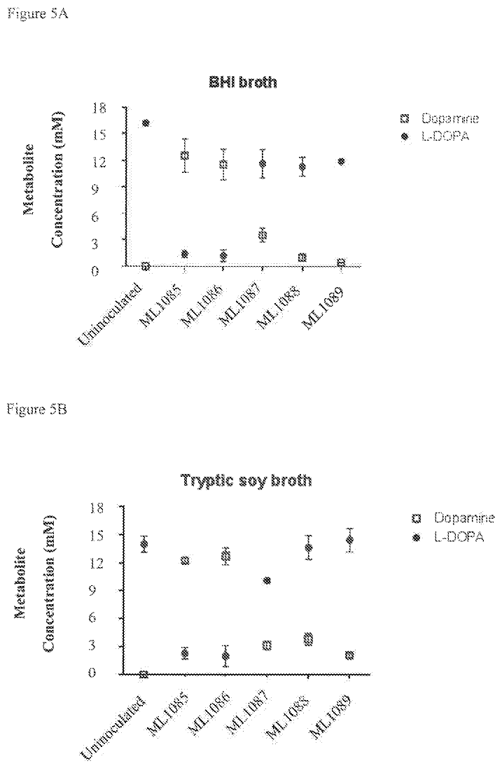

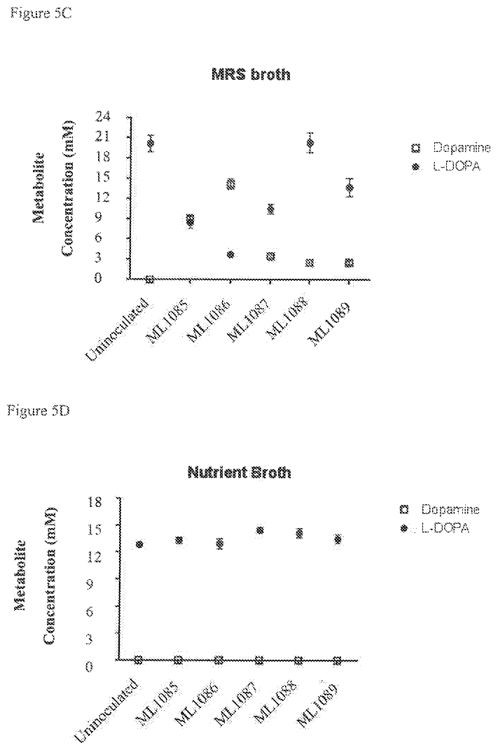

As shown in FIGS. 5A-5F, and summarized in Table 2, only strains ML1085 and ML1086 produced a significant amount of dopamine in at least one of the commercial medias, specifically: BHI broth, Tryptic soy broth, and MRS broth. No appreciable amount of dopamine was produced in Nutrient Broth, peptone water, or Luria broth by any strain. Strains ML1067, ML1068, and ML1069 produced little to not dopamine in any of the commercial medias.

Therefore, even within the same species and across different media types, the production of dopamine is highly variable. This shows the importance of screening bacteria for their ability to produce dopamine before using them for the production of dopamine, as some are better suited for production in an industrial setting.

TABLE 2

| | Unused | L-DOPA | Dopamine | |
|---|---|---|---|---|
| | L-DOPA | Consumed | Produced | Conversion |
| | (μg/mL) | (μg/mL) | (μg/mL) | Efficiency % |
| BHI ML1085 | 279 | 2922 | 1921 | 77 |
| BHI ML1086 | 239 | 2962 | 1768 | 71 |
| BHI ML1087 | 2296 | 905 | 547 | 22 |
| BHI ML1088 | 2230 | 971 | 155 | 6 |
| BHI ML1089 | 2351 | 850 | 71 | 3 |
| TSB ML1085 | 454 | 2303 | 1875 | 88 |
| TSB ML1086 | 396 | 2361 | 1949 | 91 |
| TSB ML1087 | 1988 | 770 | 483 | 23 |
| TSB ML1088 | 2690 | 67 | 585 | 27 |
| TSB ML1089 | 2658 | 100 | 294 | 14 |
| MRS ML1085 | 1673 | 2299 | 1392 | 45 |
| MRS ML1086 | 728 | 3244 | 2173 | 70 |
| MRS ML1087 | 2062 | 1910 | 531 | 17 |
| MRS ML1088 | 3734 | 238 | 382 | 12 |
| MRS ML1089 | 2711 | 1261 | 394 | 13 |

### Example 5

As dopamine may be easily oxidized in certain situations, the dopamine produced in MRS in an anaerobic environment, similar to Example 1 above, was assayed to determine if the process caused oxidation to the dopamine.

To prepare oxidized products of dopamine; a 10 mM solution of dopamine was prepared in a 2N NaOH solution. Almost immediately, the solution began turning brown, indicative of dopamine oxidation. At a pH >9.5, dopamine autoxidation occurs rapidly. The reaction was allowed to reach equilibrium and following the reaction, a 10 μL sample was screened for absorbance between the wavelengths of 190 nm and 900 nm at 2 nm increments. Strong absorbance occurred between 190 nm and 310 nm. To improve specificity, the sample was evaluated at 225 nm, 250 nm, 275 nm and 295 nm by HPLC-UV and compared to a sample of dopamine preserved by acid. Oxidized species were detectable under all wavelengths tested, but 250 nm proved more sensitive for the detection of oxidized species. Retention times of oxidized species and dopamine did not overlap thus specificity would be high even at wavelengths common to both species. Peaks unique to the oxidized sample were tagged as oxidized species related to dopamine degradation.

To test for oxidation, 10 mL of MRS media was supplemented with L-DOPA to achieve an L-DOPA concentration of 3.8 mM. Supplemented media was inoculated with 100μL of a 0.2 OD solution of E. faecium and allowed to grow overnight aerobically at 37° C.

At 24 hours, the products were processed according to Example 1 for catecholamine analysis. Briefly, samples were acidified with the addition of 10 μL 10N HCl per milliliter sample and centrifuged to remove cells and insoluble precipitates. Supernatants were then screened by HPLC-UV at 250 nm.

For a side by side comparison, oxidized products of dopamine were generated according to the above oxidation procedure at a concentration of 3.8 mM. 3.8 mM was chosen because this is the maximum theoretical amount of dopamine that can be produced by decarboxylation alone in a media which supplies 3.8 mM of L-DOPA. To achieve this, 36 mg of dopamine hydrochloride (MW: 189.64) was dissolved in 50 mL of a 2N NaOH solution to create a 3.8 mM solution of dopamine. This reaction was allowed to proceed overnight to reach equilibrium.

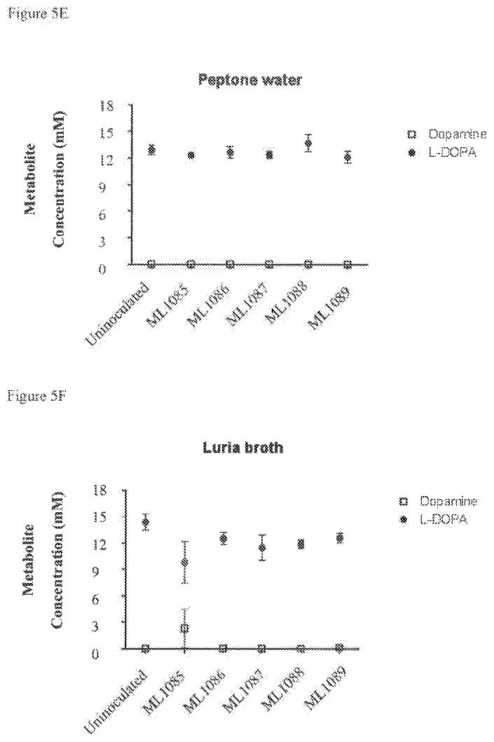

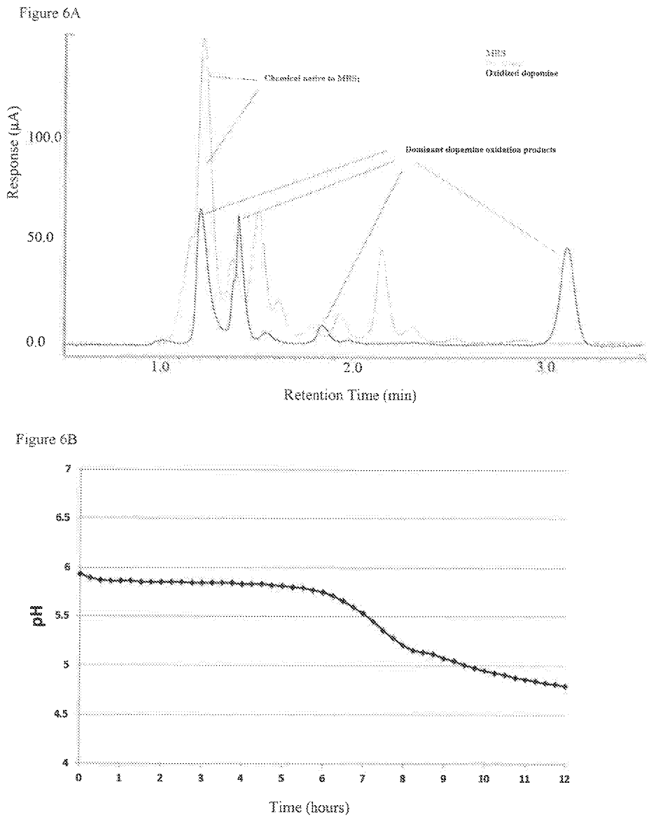

A compilation of chromatograms was created to compare uninoculated MRS, oxidized species of dopamine and an E. faecium cultured in MRS in which dopamine was produced from 3.8 mM of L-dopa (FIG. 6A). There is minimal overlap between peaks unique to oxidation and peaks generated by culture. It should be noted that overlap exists in the early portion of the chromatogram in FIG. 6A; in particular, a peak with a retention time of 1.23 minutes which overlaps with the oxidation peak with a retention time of 1.21 minutes. Additionally, an overlapping peak exists at 1.38 minutes. However, both of these peaks are natively found in MRS and do not result from the microbial activity within the culture. The peak at 1.23 minutes actually decreases following culture.

Chromatographic evaluation shows that E. faecium, performed in this manner, is free of many of the species encountered by the oxidation of dopamine. This is expected because the decarboxylation of L-DOPA under these conditions proceeds in an acidic environment as Enterococcus generates acidic metabolites (FIG. 6B). As shown in FIG. 6B, the average pH for the 12 hour period is 5.49 with a substantial drop between 6-9 hours. By 12 hours the pH has reached a pH of 4.79. If allowed to continue, the pH will continue to drop to a pH of 4.2 before the low pH and accumulation of waste metabolites results in cellular dormancy (data not shown).

Enterococci belong to a group of bacteria termed lactic acid bacteria; named for their tendency to generate lactic acid during growth. This is relevant because the auto-oxidation of dopamine is strongly dependent on pH. For example, at a pH of 5.6, the reaction rate for the auto-oxidation of dopamine is k=0.000000591 sec⁻¹ with a half-life of 13.5 days. In contrast, commonly engineered organisms like Escherichia coli, which are not considered lactic acid bacteria, do not typically thrive under acidic conditions. In fact, the media commonly used to grow E. coli, Luria broth, typically has a pH of between 7-7.5. At a pH of 7.4, the oxidation rate of dopamine is 0.000586 sec⁻¹ with a half-life of 4.95 minutes. Any dopamine produced in such an environment would be difficult to collect and process before its rapid oxidation. As an added benefit, the acidic environment of the media also favors optimal enzyme activity. For example, in the MRS described above, the pH averages 5.49 over the first 12 hours of growth (FIG. 6B). The optimal enzyme activity of tyrosine decarboxylase has been determined to be 5.5.

Therefore, this evidence shows that this method does not generate significant quantities of oxidized species derived from dopamine.

### Example 6

Following the finding that probiotic strains of E. faecium were able to produce dopamine in in vitro cultures, the genetic elements responsible for dopamine production were then identified. A BLAST search was conducted against the enterococcus genome and looked for sequences that shared similarity with known L-DOPA decarboxylases. Several searches returned hits on tyrosine decarboxylase, albeit with surprisingly low identity scores of around 20-30%.

Additionally, in order to determine the sequence of the aromatic decarboxylases across bacterial strains, several strains were sequenced using Next Generation Sequencing. E. faecium strains ML1082, ML1085, ML1086, ML1087, ML1088, and ML1089 were sequenced on an Illumina MiSeq (see Table 4 in Example 8). Following sequencing, the genomes were assembled using SPAdes and annotated using Patric using the standard procedures (see SPAdes 3.11.1 Manual, available at http://spades.bioinfspbau.ru/release3.11.1/manual.html; Bankevich A., et al., SPAdes: a new genome assembly algorithm and its application to single-cell sequencing, J Comput Biol, 2012;19(5):455-77; and Wattam A. R., et al., Improvements to PATRIC, the all-bacterial Bioinformatics Database and Analysis Resource Center, Nucleic Acids Res, 2017; 45(D1):D535-D542, all herein incorporated by reference).

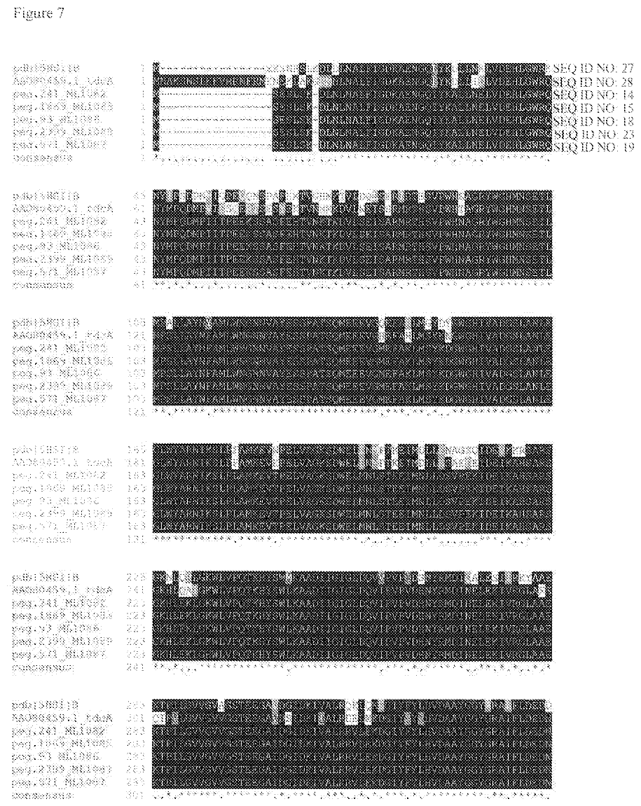

The putative aromatic decarboxylases of the strains were then aligned across the different strains as well as to Lactobacillus brevis tyrosine decarboxylase and Streptococcus. faecalis tyrosine decarboxylase using MAFFT (see Katoh et al., MAFFT online service: multiple sequence alignment, interactive sequence choice and visualization, Briefings in Bioinformatics, 2017; and Kuraku et al., aLeaves facilitates on-demand exploration of metazoan gene family trees on MAFFT sequence alignment server with enhanced interactivity, Nucleic Acids Research; 41:W22-W28, 2013, both herein incorporated by reference) and shaded using Boxshade, available on ExPASy (see FIG. 7).

The alignment shows noticeable differences between the aromatic decarboxylases found in Enterococcus faecium and both of Lactobacillus brevis and Streptococcus faecalis. This indicates that there may be variation between the substrate preference or catalytic ability between the three species of bacterium.

### Example 7

To confirm the hypothesis of this region being involved with the production of dopamine, the region containing the tyrDC gene was isolated and transformed into a microbe which was otherwise incapable of significant dopamine production. If the expression of the TyrDC enzyme in a non-native species also introduced the capacity to produce dopamine, then the ability for this enzyme to decarboxylate L-DOPA to form of dopamine would be confirmed. In reviewing the literature, it was also learned that the tyrDC gene usually exists on a gene island; with reported variations all including the gene tryP. This gene encodes for a protein associated with the extracellular export of tyramine and import of tyrosine. Recognizing that tyramine and dopamine are very similar and that the ability to export waste metabolites and import fresh substrate would be a required factor for an organism to produce dopamine, we opted to include both genes in the transformation experiments. The enzyme tryP is not a decarboxylase enzyme and is not responsible for dopamine production. However, as evidenced by its conserved location on the tyrDC gene island, it is an important coenzyme.

DNA encoding the decarboxylase enzyme tyrosine decarboxylase (tyrDC) and the enzyme tyrosine/tyramine permease (tyrP) was isolated from the E. faecium ML1082. High quality genomic DNA was isolated from the overnight grown Enterococcus faecium broth culture ML1082 using MasterPureTM Complete DNA and RNA Purification Kit (Epicentre; Cat#MC85200) by following the manufacturer's protocol. The quality and the quantity of the genomic DNA was evaluated using NanoDrop™ One Microvolume UV-Vis Spectrophotometer (Thermo ScientificTM 840274100). Sense and Antisense phosphorylated PCR primers for the

DNA segment containing the tyrDC and tyrP genes was synthesized using the sequences obtained from the published literature (Bargossi E et. al. 2015, herein incorporated by reference).

The 5′-phosphorylated blunt end PCR/gene product was generated with 50 μL PCR reaction volume by combining 25 μL Q5® High-Fidelity 2× Master Mix, 0.5 μL of both primers, 0.5 μL genomic DNA, and 24 μL Nuclease free water with the standard Q5 cycling condition (Bio-Rad PTC-1148 MJ Mini Personal Thermal Cycler; Q5 program). Gel electrophoresis was performed to confirm the correct size of the amplified gene product and for DNA purification. The amplified tyrDC and tyrP PCR/gene product in the agarose gel was extracted using Monarch® DNA Gel Extraction Kit (Cat# T1020L) following the manufacturer's instruction and the quality and the quantity of eluted DNA was verified using NanoDrop™.

The gel purified tyrDC/tyrP PCR/gene product was inserted and ligated into pSMART®LCKan by using CloneSmart® Blunt Cloning Kit. Ligation reaction (10.0 μL total reaction volume) was set in a 1.5 mL Eppendorf tube by combining 5 μL TyrDC PCR/gene DNA (˜400 ng, blunt-ended, 5′-phosphorylated), 1.5 μL H₂O, 2.5 μL 4× CloneSmart Vector Premix (pSMART vector, ATP, buffer) and 1 μL CloneSmart DNA Ligase (2U/μL). After thorough mixing and brief centrifugation, the reaction tube was incubated at 16° C. for overnight. Transformation of pSMART® Blunt Vector containing tyrDC gene into E. coli:

After overnight incubation, the ligated pSMART® Blunt Vector was transformed using chemically competent DH5-alpha cells. Briefly, the fresh ice thawed 40 μL of competent cells were added into pre-chilled 17 mm×100 mm sterile culture tubes and mixed with 1 μL of the tyrDC DNA Ligated CloneSmart plasmid followed by 30 minutes incubation on ice. After 30 minutes incubation, the cells were placed in a 42° C. water bath for 45 seconds and immediately returned to ice for 2 minutes for heat shock. Recovery Medium (960 μL) was added into the cells and the culture tube placed in a rotating incubator at 250 rpm for 1 hour at 37 ° C. The transformed cells were plated on LB plates containing the kanamycin and incubated overnight at 37° C. Transformed clones were picked up and further grown in 5 mL LB broth with kanamycin. Plasmid DNA was isolated from these transformed clones grown in LB broth using Monarch® Plasmid Miniprep Kit (Cat# T1010L) and protocol. Restriction Enzyme digestion of plasmid DNA followed by gel electrophoresis was performed to confirm the presence of tyrDC gene in the transformed E. coli clone.

The primers used to isolate tyrDC are: sense primer/TyrS-F1: GGA GCT ATA AGT ATT AAC GGT GA (SEQ ID NO: 25) and antisense primer/NhaC-R6: CCA TAA TGA A(G/T)G T(A/G)C C(A/G)C T(A/G)A CT (SEQ ID NO: 26).

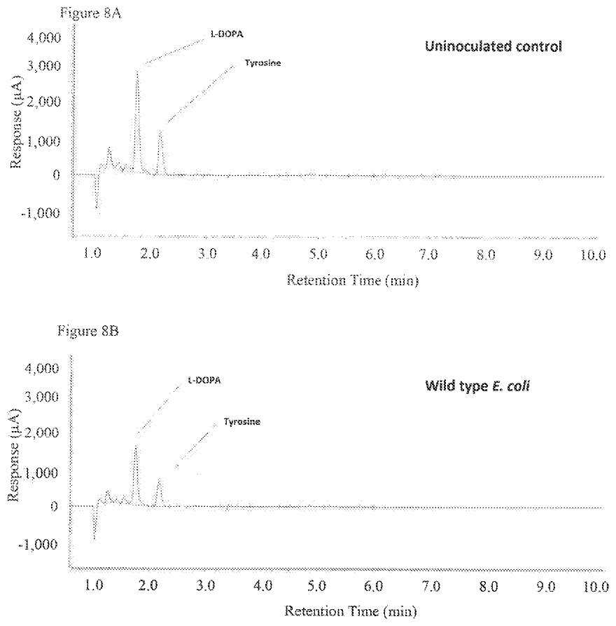

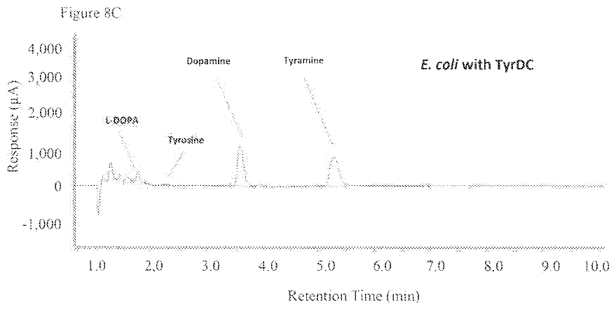

Colonies of untransformed wild type E. coli as well as the tyrDC+/tyrP+ transformed E. coli were freshly grown up on LB agar overnight, aerobically and at 37° C. to obtain metabolically active cells. Colonies were harvested and suspended in peptone water to make a 0.20D bacterial suspension. Culture broths used to validate dopamine production were prepared by inoculating 100 μL of a 0.2 OD solution into 5 mL of 1 mM L-DOPA containing sSIM. Culture broths were grown aerobically, overnight at 37° C. Conditions included control tubes which did not receive any inoculation, tubes inoculated with the wild type and tubes inoculated with tyrDC+/tyrP+transformed E. coli. Following overnight growth, 504, of 10N HCl was added to each sample. Samples were centrifuged at 3000× g for 15 minutes at 4° C. to pellet cells and insoluble precipitates. Supernatant was filtered by a 10 kDa molecular weight cut off filter. Samples were then analyzed for dopamine and L-DOPA content. Signal peak heights for tyrosine and tyramine were also reported (FIGS. 8A-8C). The results of this experiment show conclusively that the decarboxylase activity of TyrDC is responsible for the production of dopamine in Enterococcus faecium.

TABLE 3
Transformation of wild type E. coli with the genes tyrDC
and tyrP from E. faecium confers the ability to utilize
L-DOPA and tyrosine to produce both dopamine and tyramine.

| | Average | Average | Average | Average |
|---|---|---|---|---|
| | L-DOPA | dopamine | tyrosine | tyramine |
| | concentration | concentration | signal | signal |
| | (μM) | (μM) | (nA) | (nA) |
| Control | 1661 | 0 | 1011 | 0 |
| (Uninoculated) | | | | |
| Wild type | 1104 | 4 | 643 | 0 |
| tyrDC⁻/tyrP⁺ | | | | |
| E. coli | | | | |
| Transformed | 127 | 1620 | 16 | 812 |
| tyrDC⁺/tyrP⁺ | | | | |
| E. coli | | | | |

### Example 8

TABLE 4
Listing of the Sequences

| Sequence Name | Organism | Seq ID |
|---|---|---|
| ML1082 decarboxylase | E. faecium | SEQ ID NO: 1 |
| ML1082 decarboxylase | E. faecium | SEQ ID NO: 2 |
| ML1085 decarboxylase | E. faecium | SEQ ID NO: 3 |
| ML1085 decarboxylase | E. faecium | SEQ ID NO: 4 |
| ML1086 decarboxylase | E. faecium | SEQ ID NO: 5 |
| ML1086 decarboxylase | E. faecium | SEQ ID NO: 6 |
| ML1087 decarboxylase | E. faecium | SEQ ID NO: 7 |
| ML1088 decarboxylase | E. faecium | SEQ ID NO: 8 |
| ML1088 decarboxylase | E. faecium | SEQ ID NO: 9 |
| ML1088 decarboxylase | E. faecium | SEQ ID NO: 10 |
| ML1089 decarboxyase | E. faecium | SEQ ID NO: 11 |
| ML1089 decarboxyase | E. faecium | SEQ ID NO: 12 |
| ML1082 decarboxylase | E. faecium | SEQ ID NO: 13 |
| ML1082 decarboxylase | E. faecium | SEQ ID NO: 14 |
| ML1085 decarboxylase | E. faecium | SEQ ID NO: 15 |
| ML1085 decarboxylase | E. faecium | SEQ ID NO: 16 |
| ML1086 decarboxylase | E. faecium | SEQ ID NO: 17 |
| ML1086 decarboxylase | E. faecium | SEQ ID NO: 18 |
| ML1087 decarboxylase | E. faecium | SEQ ID NO: 19 |
| ML1088 decarboxylase | E. faecium | SEQ ID NO: 20 |
| ML1088 decarboxylase | E. faecium | SEQ ID NO: 21 |
| ML1088 decarboxylase | E. faecium | SEQ ID NO: 22 |
| ML1089 decarboxyase | E. faecium | SEQ ID NO: 23 |
| ML1089 decarboxyase | E. faecium | SEQ ID NO: 24 |
| tyrDC Sense Primer | Artificial | SEQ ID NO: 25 |
| tyrDC Antisense Primer | Artificial | SEQ ID NO: 26 |
| Tyrosine Decarboxylase | L. brevis | SEQ ID NO: 27 |
| Tyrosine Decarboxylase | E. faecalis | SEQ ID NO: 28 |

1. A method of producing dopamine, comprising:
contacting L-DOPA with a cell comprising an aromatic decarboxylase; and
collecting the dopamine.

2. The method of claim 1, wherein the dopamine is not oxidized.

3. The method of claim 1, wherein the L-DOPA is provided in a culture media that simulates a gastrointestinal environment.

4. (canceled)

5. The method of claim 3, wherein the culture media is simulated small intestine media.

6. The method of claim 1, wherein the L-DOPA is provided as a food source.

7. The method of claim 6, wherein the food source is an herbal or plant source.

8. (canceled)

9. The method of claim 1, wherein the microorganism is a bacterium or yeast.

10. The method of claim 9, wherein said bacteria is an Enterococcus spp., Vagococcus spp., Lactobacillus spp., Bifidobacteria spp., Carnobacterium spp., Melissococcus spp., or Escherichia spp., or wherein said yeast is a Saccharomyces spp., Candida spp., Debaryomyces spp., Kluyveromyces spp., or Yarrowia spp.

11-12. (canceled)

13. The method of claim 1, wherein the cell is a transfected eukaryote cell.

14. The method of claim 13, wherein said eukaryote cell is an insect cell, a plant cell, or a mammalian cell.

15-18. (canceled)

19. The method of claim 1, wherein said tyrosine decarboxylase is defined by one or more of: SEQ ID NOS: 1, 2, 3, 4, 5, 6, 7, 8, 9, 10, 11, or 12 with 1 or more nucleotide changes or operably linked to a heterologous promoter.

20. The method of claim 1, wherein said tyrosine decarboxylase encodes one or more proteins selected from the group consisting of: SEQ ID NOS: 13, 14, 15, 16, 17, 18, 19, 20, 21, 22, 23, or 24.

21. (canceled)

22. The method of claim 1, further comprising adding the co-factor pyridoxal phosphate.

23. The method of claim 1, further comprising:
transforming said cell with a nucleic acid molecule comprising a heterologous sequence encoding an aromatic decarboxylase operably linked to a heterologous promoter that induces transcription of said heterologous sequence in said cell.

24. The method of claim 23, wherein said heterologous sequence encodes one or more of SEQ ID NOS: 1, 2, 3, 4, 5, 6, 7, 8, 9, 10, 11, or 12 and/or wherein the cell further comprises a tyrosine permease.

25. The method of claim 23, wherein said heterologous sequence includes:
(a) a nucleotide sequence comprising the sequence set forth in SEQ ID NOS: 1, 2, 3, 4, 5, 6, 7, 8, 9, 10, 11, or 12;
(b) a nucleotide sequence comprising at least 50 contiguous nucleotides of the sequence of SEQ ID NOS: 1, 2, 3, 4, 5, 6, 7, 8, 9, 10, 11, or 12, wherein said nucleotide sequence encodes a protein capable of dopamine production;
(c) a nucleotide sequence having at least 90% sequence identity to the sequence of SEQ ID NO: 1, 2, 3, 4, 5, 6, 7, 8, 9, 10, 11, or 12;
(d) a nucleotide sequence that encodes the protein sequence of SEQ ID NO: 13, 14, 15, 16, 17, 18, 19, 20, 21, 22, 23, or 24; or
(e) a nucleotide sequence that encodes a protein sequence having at least 90% sequence identity to the sequence of SEQ ID NO: 13, 14, 15, 16, 17, 18, 19, 20, 21, 22, 23, or 24.

26. (canceled)

27. A modified cell with increased dopamine production compared to a corresponding microorganism with no such modification;
wherein said modified microorganism having a heterologous nucleotide sequence which includes a dopamine production nucleic acid sequence,
wherein said nucleic acid sequence encodes a protein selected from the group consisting of: aromatic-L-amino-acid decarboxylase, tyrosine decarboxylase, or histidine decarboxylase.

28. The modified cell of claim 27, wherein said dopamine production nucleic acid sequence: is one or more of: SEQ ID NOS: 1, 2, 35 4, 5, 6, 7, 8, 9, 10, 11, or 12 with 1 or more nucleotide changes; is one or more of SEQ ID NOS: 1, 2, 3, 4, 5, 6, 7, 8, 9, 10, 11, or 12 operably linked to a heterologous promoter; or encodes one or more proteins selected from the group consisting of: SEQ ID NOS: 13, 14, 15, 16, 17, 18, 19, 20, 21, 22, 23, or 24.

29-30. (canceled)

31. The modified cell of claim 27, further comprising a tyrosine permease.

32. An isolated nucleic acid molecule, said molecule encoding a dopamine production protein wherein said nucleic acid molecule comprises a nucleotide sequence selected from the group consisting of:
(a) a nucleotide sequence comprising the sequence set forth in SEQ ID NOS: 1, 2, 3, 4, 5, 6, 7, 8, 9, 10, 11, or 12 with one or more nucleotide changes;
(b) a nucleotide sequence comprising at least 50% contiguous nucleotides of the sequence of SEQ ID NOS: 1, 2, 3, 4, 5, 6, 7, 8, 9, 10, 11, or 12, wherein said nucleotide sequence encodes a protein for dopamine production; and
(c) a nucleotide sequence having at least 90% sequence identity to the sequence of SEQ ID NOS: 1, 2, 3, 4, 5, 6, 7, 8, 9, 10, 11, or 12, wherein said nucleotide sequence encodes a protein for dopamine production.

33-61. (canceled)


SEQUENCE LISTING (28 sequences)

SEQ ID NO: 1 - DNA, 1836 bp, Enterococcus faecium
atgaaagatatggatatcaaggccgtctttataggcgacaaagctgaaaatggaccagtttataaaatgttgttgaacaagatggtagatgaacatcttggatggagagaaaattatattccttcagatatgcctgcaattagcgaaggagataagctcactccagattatctagccacccgcgaccatatgatagaggtcttggatgaagtcagtcagcgattacgcgcaggatcgatcccatggcattcagcaggccgttactggggacaaatgaatgctgaaacattgatgcctgctttattagcttacaactatgcgatgctgtggaacccaaataatgttgcattggaatcttctatggcaacttcgcaaatggaagcagaagtaggacaggattttgcttcattattcaatatgacagacggatggggacacattgcagcagatggttctattgctaatcttgaaggattatggtacgcccgttgcatcaaatcgattcctttagctgtcaaggaagtgctgcctgaaaaagtgaaaaaaatgtctgagtgggaacttttgaatttatctgttgaagaaattttggaaatgaccgaaagctttacggacgaagaaatggatgaggtcaaagcagcttcttcccgtagcggaaaaaatatccaaagattagggaaatggctggtccctcaaacaaaacactattcttggatgaaggcgttagatatttgcggagtcggcttagatcagatggttgctatcccagttcaagaggattatcgaatggatattaatgctttggaaaaaacaattcgtgaattagctggacaaaaaatcccaattctaggtgtggttgctgtcgtaggaacgacagaagaaggtcaagtagacagtgtagataaaatcgtccaattaagagaaaggttaaaagatgaagggatctatttctatttacatgttgatgccgcatatggcggctatgcgcgttcactgtttctaaacgaagcaggagaatttgtgccgtatgcttccttagctgaattctttgaagaacatcatgttttccaccactgcgtaacgattgacaaagaggtatatgaagggttcagagctatttcagaagcggattctgtcacgatagatcctcataagatgggctatgtcccttatgctgctggaggaatcgtaatcaaacataaaaatatgagaaatatcatttcttatttcgcgccatatgtgtttgaaaaatcagtaaaggctccagatatgttaggagcttatattttggaaggatcaaaagctggagcaaccgccgccgcagtctggacagcacatcgtgtattgccattgaatgtgaccggatatggtcaattgattggggcttcaatcgaggcagctcaaagattcagagaatttctcgatcatctgacttttactgttaaaggaaaaacaatcgaagtctatccattgaatcatcctgattttaatatggtcaattgggtattcaaagaacaaggctgtacagatttgaacgctatcaatgaattaaatgaaaaaatgttcgatcggtcttcttatatggatggcgatgtttatggtgaacggtttattacttctcatactacatttacacaagaagattacggtgattctccaattcgttttgttgaaagaatgggtctaacgaaagaagaatggaaaaaagaacagaaaatcactctgctacgtgcggctatcatgactccttatttgaatgatgatcgaattttcaatttttatacaaaaaaaatcgccaaagcaatggaaaagaaactaaacgaaatcatccaatag

SEQ ID NO: 2 - DNA, 1878 bp, Enterococcus faecium
atgagtgaatcattgtcgaaagatctaaacctaaatgccctattcattggggacaaagctgagaacggccaaatctataaagctttgttgaatgaattggtggatgaacatttaggctggcgtcaaaactacatgcctcaagacatgcctattattacgccagaagaaaaaagcagtgctagttttgaacacactgtgaataaaacaaaggatgttctatcagaaatttcagcgcggatgcgtacccattccgttccttggcataatgccggacgttactggggtcatatgaactctgaaacattgatgccatctttactagcgtacaattttgctatgctatggaatggtaataacgttgcctatgaatcttctcctgcaacaagccaaatggaagaagaagtaggaatggaatttgcaaaattaatgagctataaagatggctggggccacatcgttgctgatggttctttagcaaacttagaaggactttggtatgcccgcaacatcaaatcattgccgcttgcaatgaaagaagtgacacctgaactagttgctggaaaaagcgattgggaattaatgaacttgtcaacagaagaaatcatgaatctgttagacagcgtcccagaaaaaatcgacgaaatcaaagctcactcagcacgtagcggaaaacatttagaaaaattaggaaaatggttggttccacaaactaaacactattcatggctaaaagctgccgacatcatcggtatcggtttagaccaagtgattccagtgccagttgaccacaactatcgcatggacatcaacgaacttgaaaaaatcgttcgtggtctagcagctgaaaaaacaccaatcctaggtgtagtcggtgttgtaggatcaacagaagaaggagcaatcgacggtatcgataaaatcgttgcattgcgtcgcgtattagaaaaagacggtatttacttctacttacacgtagatgctgcttatggcggatacggccgtgcgattttcttagacgaagacaacaacttcattccatttgaagatttaaaagatgttcattacaaatacaatgtctttacagaaaacaaagattatatcttagaagaagtacacagtgcatataaagcaatcgaagaagcagaatctgtaacgattgacccacataaaatgggctatgttccatattctgccggtggtatcgtcatcaaagatatccgcatgagagacgttatctcttactttgcgacatatgtatttgaaaaaggcgcagatatcccagcattacttggggcttatatcttggaaggttcaaaagcaggggcaacagcagccagcgtttgggcagcacaccatgtcttacctttgaatgtcacaggatatggtaaattaatgggtgcatcaatcgaaggtgcacatcgtttctacaacttcttgaatgacttgtcatttaaagtaggagacaaagaaatcgaagttcatccattaacttatccagatttcaatatggtagactatgtattcaaagaaaaaggcaacgatgacttagttgcaatgaataaattgaaccatgacgtatatgattattcttcatacgtgaaaggaagcatttacggtaatgagttcttaacttcacatactgactttgctatcccagactatggcaacagcccattacaatttgtgaaccaactaggattctcagatgaagaatggaaccgtgccggaaaagtaactgtattgcgtgccagcgtaatgacaccatacatgaacaaagaagaacactttgaagaatatgctgaaaaaatcaaagcagctcttcaagaaaaattggaaaaaatctacgcagaccaattattggcaagcgaagcaaaataa

SEQ ID NO: 3 - DNA, 1878 bp, Enterococcus faecium; identical to SEQ ID NO: 2

SEQ ID NO: 4 - DNA, 1836 bp, Enterococcus faecium
atgaaagatatggatatcaaggccgtctttataggcgacaaagctgaaaatggaccagtttataaaatgttgttgaacaagatggtagatgaacatcttggatggagagaaaattatattccttcagatatgcctgcaattagcgaaggagataagctcactccagattatctagccacccgcgaccatatgatagaggtcttggatgaagtcagtcagcgattacgcgcaggatcgatcccatggcattcagcaggccgttactggggacaaatgaatgctgaaacattgatgcctgctttattagcttacaactatgcgatgctgtggaacccaaataatgttgcattggaatcttctatggcaacttcgcaaatggaagcagaagtaggacaggattttgcttcattattcaatatgacagacggatggggacacattgcagcagatggttctattgctaatcttgaaggattatggtacgcccgttgcatcaaatcgattcctttagctgtcaaggaagtgctgcctgaaaaagtgaaaaaaatgtctgagtgggaacttttgaatttatctgttgaagaaattttggaaatgaccgaaagctttacggacgaagaaatggatgaggtcaaagcagcttcttcccgtagcggaaaaaatatccaaagattagggaaatggctggtccctcaaacaaaacactattcttggatgaaggcgttagatatttgcggagtcggcttagatcagatggttgctatcccagttcaagaggattatcgaatggatattaatgctttggaaaaaacaattcgtgaattagctggacaaaaaatcccaattctaggtgtggttgctgtcgtaggaacgacagaagaaggtcaagtagacagtgtagataaaatcgtccaattaagagaaaggttaaaagatgaagggatctatttctatttacatgttgatgccgcatatggcggctatgcgcgttcactgtttctaaacgaagcaggagaatttgtgccgtatgcttccttagctgaattctttgaagaacatcatgttttccaccactgcgtaacgattgacaaagaggtatatgaagggttcagagctatttcagaagcggattctgtcacgatagatcctcataagatgggctatgtcccttatgctgctggaggaatcgtaatcaaacataaaaatatgagaaatatcatttcttatttcgcgccatatgtgtttgaaaaatcagtaaaggctccagatatgttaggagcttatattttggaaggatcaaaagctggagcaaccgccgccgcagtctggacagcacatcgtgtattgccattgaatgtgaccggatatggtcaattgattggggcttcaatcgaggcagctcaaagattcagagaatttctcgatcatctgacttttactgttaaaggaaaaacaatcgaagtctatccattgaatcatcctgattttaatatggtcaattgggtattcaaagaacaaggctgtacagatttgaacgctatcaatgaattaaatgaaaaaatgttcgatcggtcttcttatatggatggcgatgtttatggtgaacggtttattacttctcatactacatttacacaagaagattacggtgattctccgattcgttttgttgaaagaatgggtctaacgaaagaagaatggaaaaaagaacagaaaatcactctgctacgtgcggctatcatgactccttatttgaatgatgatcgaattttcaatttttatacaaaaaaaatcgccaaagcaatggaaaagaaactaaacgaaatcatccaatag

SEQ ID NO: 5 - DNA, 1836 bp, Enterococcus faecium; identical to SEQ ID NO: 4

SEQ ID NO: 6 - DNA, 1878 bp, Enterococcus faecium; identical to SEQ ID NO: 2

SEQ ID NO: 7 - DNA, 1878 bp, Enterococcus faecium
atgagtgaatcattgtcgaaagatctaaacctaaatgccctattcattggggacaaagctgagaacggccaaatctataaagctttgttgaatgaattggtggatgaacatttaggctggcgtcaaaactacatgcctcaagacatgcctattattacgccagaagaaaaaagcagtgctagttttgaacacactgtgaataaaacaaaggatgttctatcagaaatttcagcgcggatgcgtacccattccgttccttggcataatgccggacgttactggggtcatatgaactctgaaacattgatgccatctttactagcgtacaattttgctatgctatggaatggtaataacgttgcctatgaatcttctcctgcaacaagccaaatggaagaagaagtaggaatggaatttgcaaaattaatgagctataaagatggctggggccacatcgttgctgatggttctttagcaaacttagaaggactttggtatgcccgcaacatcaaatcattgccgcttgcaatgaaagaagtgacacctgaactagttgctggaaaaagcgattgggaattaatgaacttgtcaacagaagaaatcatgaatctgttagacagcgtcccagaaaaaatcgacgaaatcaaagctcactcagcacgtagcggaaaacatttagaaaaattaggaaaatggttggttccacaaactaaacactattcatggctaaaagctgccgacatcatcggtatcggtttagaccaagtgattccagtgccagttgaccacaactatcgcatggacatcaacgaacttgaaaaaatcgttcgtggtctagcagctgaaaaaacaccaatcctaggtgtagtcggtgttgtaggatcaacagaagaaggagcaatcgacggtatcgataaaatcgttgcattgcgtcgcgtattagaaaaagacggtatttacttctacttacacgtagatgctgcttatggcggatacggccgtgcgattttcttagacgaagacaacaacttcattccatttgaagatttaaaagatgttcattacaaatacaatgtctttacagaaaacaaagattatatcttagaagaagtacacagtgcatataaagcaatcgaagaagcagaatctgtaacgattgacccacataaaatgggctatgttccatattctgccggtggtatcgtcatcaaagatatccgcatgagagacgttatctcttactttgcgacatatgtatttgaaaaaggcgcagatatcccagcattacttggggcttatatcttggaaggttcaaaagcaggggcaacagcagccagcgtttgggcagcacaccatgtcttacctttgaatgtcacaggatatggtaaattaatgggtgcatcaatcgaaggtgcacatcgtttctacaacttcttgaatgacttgtcatttaaagtaggagacaaagaaatcgaagttcatccattaacttatccagatttcaatatggtagactatgtattcaaagaaaaaggcaacgatgacttagttgcaatgaataaattgaaccatgacgtatatgattattcttcatacgtgataggaagcatttacggtaatgagttcttaacttcacatactgactttgctatcccagactatggcaacagcccattacaatttgtgaaccaactaggattctcagatgaagaatggaaccgtgccggaaaagtaactgtattgcgtgccagcgtaatgacaccatacatgaacaaagaagaacactttgaagaatatgctgaaaaaatcaaagcagctcttcaagaaaaattggaaaaaatctacgcagaccaattattggcaagcgaagcaaaataa

SEQ ID NO: 8 - DNA, 1746 bp, Enterococcus faecium
atgcctcaagacatgcctattattacgccagaagaaaaaagcagtgctagttttgaacacactgtgaataaaacaaaggatgttctatcagaaatttcagcgcggatgcgtacccattccgttccttggcataatgccggacgttactggggtcatatgaactctgaaacattgatgccatctttactagcgtacaattttgctatgctatggaatggtaataacgttgcctatgaatcttctcctgcaacaagccaaatggaagaagaagtaggaatggaatttgcaaaattaatgagctataaagatggctggggccacatcgttgctgatggttctttagcaaacttagaaggactttggtatgcccgcaacatcaaatcattgccgcttgcaatgaaagaagtgacacctgaactagttgctggaaaaagcgattgggaattaatgaacttgtcaacagaagaaatcatgaatctgttagacagcgtcccagaaaaaatcgacgaaatcaaagctcactcagcacgtagcggaaaacatttagaaaaattaggaaaatggttggttccacaaactaaacactattcatggctaaaagctgccgacatcatcggtatcggtttagaccaagtgattccagtgccagttgaccacaactatcgcatggacatcaacgaacttgaaaaaatcgttcgtggtctagcagctgaaaaaacaccaatcctaggtgtagtcggtgttgtaggatcaacagaagaaggagcaatcgacggtatcgataaaatcgttgcattgcgtcgcgtattagaaaaagacggtatttacttctacttacacgtagatgctgcttatggcggatacggccgtgcgattttcttagacgaagacaacaacttcattccatttgaagatttaaaagatgttcattacaaatacaatgtctttacagaaaacaaagattatatcttagaagaagtacacagtgcatataaagcaatcgaagaagcagaatctgtaacgattgacccacataaaatgggctatgttccatattctgccggtggtatcgtcatcaaagatatccgcatgagagacgttatctcttactttgcgacatatgtatttgaaaaaggcgcagatatcccagcattacttggggcttatatcttggaaggttcaaaagcaggggcaacagcagccagcgtttgggcagcacaccatgtcttacctttgaatgtcacaggatatggtaaattaatgggtgcatcaatcgaaggtgcacatcgtttctacaacttcttgaatgacttgtcatttaaagtaggagacaaagaaatcgaagttcatccattaacttatccagatttcaatatggtagactatgtattcaaagaaaaaggcaacgatgacttagttgcaatgaataaattgaaccatgacgtatatgattattcttcatacgtgaaaggaagcatttacggtaatgagttcttaacttcacatactgactttgctatcccagactatggcaacagcccattacaatttgtgaaccaactaggattctcagatgaagaatggaaccgtgccggaaaagtaactgtattgcgtgccagcgtaatgacaccatacatgaacaaagaagaacactttgaagaatatgctgaaaaaatcaaagcagctcttcaagaaaaattggaaaaaatctacgcagaccaattattggcaagcgaagcaaaataa

SEQ ID NO: 9 - DNA, 120 bp, Enterococcus faecium
atgagtgaatcattgtcgaaagatctaaacctaaatgccctattcattggggacaaagctgagaacggccaaatctataaagctttgttgaatgaattggtggatgaacatttaggctga

SEQ ID NO: 10 - DNA, 1836 bp, Enterococcus faecium; identical to SEQ ID NO: 4

SEQ ID NO: 11 - DNA, 1878 bp, Enterococcus faecium
atgagtgaatcattgtcgaaagatctaaacctaaatgccctattcattggggacaaagctgagaacggccaaatctataaagctttgttgaatgaattggtggacgaacatttaggctggcgtcaaaactacatgcctcaagacatgcctatcattacgccagaagaaaaaagcagtgctagttttgaacacactgtgaataaaacaaaagatgttctatcagaaatttcagcgcggatgcgtacccattccgttccttggcataatgccggacgctactggggtcatatgaactctgaaacattgatgccatctttactagcgtacaattttgctatgctatggaatggtaataacgttgcttacgaatcttctcctgcaacaagccaaatggaagaagaagtaggcatggaatttgcaaaattaatgagctataaagatggctggggacacatcgttgctgatggttctttagcaaacttagaaggactttggtatgcccgcaacatcaaatcattgccacttgctatgaaagaagtgacacctgagctagttgctggaaaaagcgattgggaattaatgaacttgtcaacagaagaaatcatgaacttgttagacagtgttccagaaaaaatcgacgaaatcaaagctcactcagcacgtagcggaaaacatttagaaaaattaggaaaatggttggttccacaaacaaaacactattcatggctaaaagctgccgacatcatcggtatcggtttagaccaagtaattccagtgccagttgaccataactatcgcatggacatcaacgaacttgaaaaaatcgttcgtggtctagcagctgaaaaaacaccaatcttaggtgtagtcggtgttgtaggatcaacagaagaaggagcaatcgacggtatcgataaaatcgttgcattgcgtcgcgtattagaaaaagacggtatttacttctacttgcacgtagatgctgcttatggcggatatggccgtgcgattttcttagacgaagacaacaacttcattccattcgaagatttaaaagatgttcattacaaatacaacgtctttacagaaaacaaagattatatcttagaagaagtacacagtgcatataaagcaatcgaagaagcagaatctgtaacgatcgacccacataaaatgggctatgttccatattctgccggtggtatcgtcatcaaagatatccgcatgagagacgttatctcttacttcgcaacatatgtatttgaaaaaggtgcagatatcccagcattacttggagcgtatatcttggaaggttcaaaagcaggggcaacagcagccagcgtttgggcagcacatcatgtcttacctttgaatgtcacaggatacggtaaattgatgggtgcatcaatcgaaggggcacatcgtttctacaacttcttgaatgacttgtcattcaaagtaggtgacaaagaaatcgaagttcatccattaacttatccagatttcaacatggtagactacgtattcaaagaaaaaggcaacgacgatttagttgcgatgaataaattgaaccatgacgtatacgattattcttcatacgtgaaaggaagcatttacggtaatgaattcttaacttcacatactgactttgctatcccagactatggcaacagcccattgcaattcgtgaaccaactaggattctcagatgaagaatggaaccgtgccggaaaagtaactgtattgcgtgccagcgtaatgacaccatacatgaacaaagaagaacactttgaagaatatgctgaaaaaatcaaagcagctcttcaagaaaaattggaaaaaatctacgcagaccaattattggcaagcgaagcaaaataa

SEQ ID NO: 12 - DNA, 1766 bp, Enterococcus faecium
tgttgaacaagatggtagatgaacatcttggctggagagaaaattatattccttcagatatgcctgcaattagcgaaggagataagctaactccagattatctagccacccgcgaccatatgatagaggtcttggatgaagtcagtcagcgattacgcgcaggatcaatcccatggcattcagcaggccgttactggggacaaatgaacgctgaaacactgatgcctgctttattagcttacaattatgcgatgctatggaatcctaataacgttgcactagaatcttctatggcaacttcgcagatggaagcagaagtaggacaggatttagcctcactattcaacatggcagacggatggggacatatcgcagcagatggttctattgctaaccttgaaggattatggtacgcccgttgcatcaaatcgattcctttagctgtcaaggaagttttgcctgaaaaagtgaaaaacatgtctgagtgggctcttttgaatctgtctgttgaagaaattttggaaatgaccgaaagctttacagatgaagagatggatgaggtcaaagcagcttcttctcgtagcggaaagaatatccaaaaattagggaaatggctggttcctcaaacaaaacactattcttggatgaaggcgttagatatttgcggagtcggcttagatcagatggttgctatcccagttcaagaggattatcgaatggatatcaacgctttggaaaaaacaattcgtgaattagctgatcaaaaaatcccgattctaggtgtggttgctgtcgtaggaacaacagaagaaggacaagtagacagtgtagataaaatcattcggttaagagaaaagttaaaagatgaaggtatctatttctatttacatgttgacgctgcatatggcgggtacgcgcgttcactgtttttaaacgaagcaggagaatttgtgccatatgcttccttagctgaattctttgaagagcatcatgtattccaccattacgtaacgatcgacaaagaggtgtatgaaggattcagagctatttcagaagcggattctgtcacgattgatccgcataagatgggctatgtcccttatgctgctggaggaatcgtgatcaaacataaaaatatgagaaatatcatttcttattttgcaccatatgtgtttgaaaaatcagtcaaagcaccagatatgctaggtgcctatattttagaaggctcaaaagcaggagcaacggctgctgcagtctggacagctcatcgtgtattgccattgaatgtgaccggatatggtcaattgattggtgcttcaatcgaagcagctcaaagattcagagaatttttaggtcaattacactttactgttaaaggaaaaataattgaagtctatccactgaatcatcctgattttaatatggtcaattgggtatttaaagtacaagattgtacagatttgaacgctatcaatgaattaaatgaaaaaatgttcgatcggtcttcttatatggatggcgacgtttatggtgaacggtttattacttctcatacgacattcacacaagaagattacggtgattctccgatccgttttattgaaagaatgggtctatcgaaagaagaatggcaaaaagaacagcaaatcactctgctacgtgcggctatcatgactccttatttgaatgatgatcgaattttcaatttttatacaaaagaaatcgccaaagcaatggaaaagaaactaaacgaaatcatcaaatag

SEQ ID NO: 13 - protein, 611 aa, Enterococcus faecium
MKDMDIKAVFIGDKAENGPVYKMLLNKMVDEHLGWRENYIPSDMPAISEGDKLTPDYLATRDHMIEVLDEVSQRLRAGSIPWHSAGRYWGQMNAETLMPALLAYNYAMLWNPNNVALESSMATSQMEAEVGQDFASLFNMTDGWGHIAADGSIANLEGLWYARCIKSIPLAVKEVLPEKVKKMSEWELLNLSVEEILEMTESFTDEEMDEVKAASSRSGKNIQRLGKWLVPQTKHYSWMKALDICGVGLDQMVAIPVQEDYRMDINALEKTIRELAGQKIPILGVVAVVGTTEEGQVDSVDKIVQLRERLKDEGIYFYLHVDAAYGGYARSLFLNEAGEFVPYASLAEFFEEHHVFHHCVTIDKEVYEGFRAISEADSVTIDPHKMGYVPYAAGGIVIKHKNMRNIISYFAPYVFEKSVKAPDMLGAYILEGSKAGATAAAVWTAHRVLPLNVTGYGQLIGASIEAAQRFREFLDHLTFTVKGKTIEVYPLNHPDFNMVNWVFKEQGCTDLNAINELNEKMFDRSSYMDGDVYGERFITSHTTFTQEDYGDSPIRFVERMGLTKEEWKKEQKITLLRAAIMTPYLNDDRIFNFYTKKIAKAMEKKLNEIIQ

SEQ ID NO: 14 - protein, 625 aa, Enterococcus faecium
MSESLSKDLNLNALFIGDKAENGQIYKALLNELVDEHLGWRQNYMPQDMPIITPEEKSSASFEHTVNKTKDVLSEISARMRTHSVPWHNAGRYWGHMNSETLMPSLLAYNFAMLWNGNNVAYESSPATSQMEEEVGMEFAKLMSYKDGWGHIVADGSLANLEGLWYARNIKSLPLAMKEVTPELVAGKSDWELMNLSTEEIMNLLDSVPEKIDEIKAHSARSGKHLEKLGKWLVPQTKHYSWLKAADIIGIGLDQVIPVPVDHNYRMDINELEKIVRGLAAEKTPILGVVGVVGSTEEGAIDGIDKIVALRRVLEKDGIYFYLHVDAAYGGYGRAIFLDEDNNFIPFEDLKDVHYKYNVFTENKDYILEEVHSAYKAIEEAESVTIDPHKMGYVPYSAGGIVIKDIRMRDVISYFATYVFEKGADIPALLGAYILEGSKAGATAASVWAAHHVLPLNVTGYGKLMGASIEGAHRFYNFLNDLSFKVGDKEIEVHPLTYPDFNMVDYVFKEKGNDDLVAMNKLNHDVYDYSSYVKGSIYGNEFLTSHTDFAIPDYGNSPLQFVNQLGFSDEEWNRAGKVTVLRASVMTPYMNKEEHFEEYAEKIKAALQEKLEKIYADQLLASEAK

SEQ ID NO: 15 - protein, 625 aa, Enterococcus faecium; identical to SEQ ID NO: 14

SEQ ID NO: 16 - protein, 611 aa, Enterococcus faecium; identical to SEQ ID NO: 13

SEQ ID NO: 17 - protein, 611 aa, Enterococcus faecium; identical to SEQ ID NO: 13

SEQ ID NO: 18 - protein, 625 aa, Enterococcus faecium; identical to SEQ ID NO: 14

SEQ ID NO: 19 - protein, 625 aa, Enterococcus faecium
MSESLSKDLNLNALFIGDKAENGQIYKALLNELVDEHLGWRQNYMPQDMPIITPEEKSSASFEHTVNKTKDVLSEISARMRTHSVPWHNAGRYWGHMNSETLMPSLLAYNFAMLWNGNNVAYESSPATSQMEEEVGMEFAKLMSYKDGWGHIVADGSLANLEGLWYARNIKSLPLAMKEVTPELVAGKSDWELMNLSTEEIMNLLDSVPEKIDEIKAHSARSGKHLEKLGKWLVPQTKHYSWLKAADIIGIGLDQVIPVPVDHNYRMDINELEKIVRGLAAEKTPILGVVGVVGSTEEGAIDGIDKIVALRRVLEKDGIYFYLHVDAAYGGYGRAIFLDEDNNFIPFEDLKDVHYKYNVFTENKDYILEEVHSAYKAIEEAESVTIDPHKMGYVPYSAGGIVIKDIRMRDVISYFATYVFEKGADIPALLGAYILEGSKAGATAASVWAAHHVLPLNVTGYGKLMGASIEGAHRFYNFLNDLSFKVGDKEIEVHPLTYPDFNMVDYVFKEKGNDDLVAMNKLNHDVYDYSSYVIGSIYGNEFLTSHTDFAIPDYGNSPLQFVNQLGFSDEEWNRAGKVTVLRASVMTPYMNKEEHFEEYAEKIKAALQEKLEKIYADQLLASEAK

SEQ ID NO: 20 - protein, 581 aa, Enterococcus faecium
MPQDMPIITPEEKSSASFEHTVNKTKDVLSEISARMRTHSVPWHNAGRYWGHMNSETLMPSLLAYNFAMLWNGNNVAYESSPATSQMEEEVGMEFAKLMSYKDGWGHIVADGSLANLEGLWYARNIKSLPLAMKEVTPELVAGKSDWELMNLSTEEIMNLLDSVPEKIDEIKAHSARSGKHLEKLGKWLVPQTKHYSWLKAADIIGIGLDQVIPVPVDHNYRMDINELEKIVRGLAAEKTPILGVVGVVGSTEEGAIDGIDKIVALRRVLEKDGIYFYLHVDAAYGGYGRAIFLDEDNNFIPFEDLKDVHYKYNVFTENKDYILEEVHSAYKAIEEAESVTIDPHKMGYVPYSAGGIVIKDIRMRDVISYFATYVFEKGADIPALLGAYILEGSKAGATAASVWAAHHVLPLNVTGYGKLMGASIEGAHRFYNFLNDLSFKVGDKEIEVHPLTYPDFNMVDYVFKEKGNDDLVAMNKLNHDVYDYSSYVKGSIYGNEFLTSHTDFAIPDYGNSPLQFVNQLGFSDEEWNRAGKVTVLRASVMTPYMNKEEHFEEYAEKIKAALQEKLEKIYADQLLASEAK

SEQ ID NO: 21 - protein, 39 aa, Enterococcus faecium
MSESLSKDLNLNALFIGDKAENGQIYKALLNELVDEHLG

SEQ ID NO: 22 - protein, 611 aa, Enterococcus faecium; identical to SEQ ID NO: 13

SEQ ID NO: 23 - protein, 625 aa, Enterococcus faecium; identical to SEQ ID NO: 14

SEQ ID NO: 24 - protein, 611 aa, Enterococcus faecium
MKDMDIKAVFIGDKAENGPVYKMLLNKMVDEHLGWRENYIPSDMPAISEGDKLTPDYLATRDHMIEVLDEVSQRLRAGSIPWHSAGRYWGQMNAETLMPALLAYNYAMLWNPNNVALESSMATSQMEAEVGQDLASLFNMADGWGHIAADGSIANLEGLWYARCIKSIPLAVKEVLPEKVKNMSEWALLNLSVEEILEMTESFTDEEMDEVKAASSRSGKNIQKLGKWLVPQTKHYSWMKALDICGVGLDQMVAIPVQEDYRMDINALEKTIRELADQKIPILGVVAVVGTTEEGQVDSVDKIIRLREKLKDEGIYFYLHVDAAYGGYARSLFLNEAGEFVPYASLAEFFEEHHVFHHYVTIDKEVYEGFRAISEADSVTIDPHKMGYVPYAAGGIVIKHKNMRNIISYFAPYVFEKSVKAPDMLGAYILEGSKAGATAAAVWTAHRVLPLNVTGYGQLIGASIEAAQRFREFLGQLHFTVKGKIIEVYPLNHPDFNMVNWVFKVQDCTDLNAINELNEKMFDRSSYMDGDVYGERFITSHTTFTQEDYGDSPIRFIERMGLSKEEWQKEQQITLLRAAIMTPYLNDDRIFNFYTKEIAKAMEKKLNEIIK

SEQ ID NO: 25 - DNA, 23 bp, Artificial Sequence
  tyrDC Primer
ggagctataagtattaacggtga

SEQ ID NO: 26 - DNA, 23 bp, Artificial Sequence
  tyrDC Primer
ccataatgaakgtrccrctract

SEQ ID NO: 27 - protein, 634 aa, Lactobacillus brevis
MEKSNRSLKDLDLNALFIGDKAENGQLYKDLLNKLVDEHLGWRKNYIPSDPNMIGPEDQNSPAFKKTVGHMKTVLDQLSERIRTESVPWHSAGRYWGHMNSETLMPALLAYNYAMLWNGNNVAYESSPATSQMEEEVGQEFARLMGYDYGWGHIVADGSLANLEGLWYARNIKSLPFAMKEVNPELVAGKSDWELLNMPTKEIMDLLENAGSQIDEVKKRSARSGKNLQRLGKWLVPQTKHYSWMKAADIIGIGLDQVVPVPIDSNYRMDIQALESIIRKYAAEKTPILGVVGVAGSTEEGAVDGIDKIVALRQKLQKEGIYFYLHVDAAYGGYARALFLDEDDQFIPYKNLQKVHAENHVFTEDKEYIKPEVYAAYKAFDQAESITIDPHKMGYVPYSAGGIVIQDIRMRDTISYFATYVFEKGADIPALLGAYILEGSKAGATAASVWAAHHTLPLNVTGYGKLEGASIEGAHRYYDFLKNLKFEVAGKRISVHPLISPDFNMVDYVLKEDGNDDLIEMNRLNHAFYEQASYVKGSLYGKEYIVSHTDFAIPDYGDSPLAFVESLGFSEVEWRHAGKVTIIRASVMTPYMNQRENFDYFAPRIKKAIQADLEKVYASVNQKENVLEHHHHHH

SEQ ID NO: 28 - protein, 636 aa, Enterococcus faecalis
MNAKSNSLKFVRRNFRMKNEKLAKGEMNLNALFIGDKAENGQLYKDLLIDLVDEHLGWRQNYMPQDMPVISSQERTSESYEKTVNHMKDVLNEISSRMRTHSVPWHTAGRYWGHMNSETLMPSLLAYNFAMLWNGNNVAYESSPATSQMEEEVGHEFAHLMSYKNGWGHIVADGSLANLEGLWYARNIKSLPFAMKEVKPELVAGKSDWELLNMPTKEIMDLLESAEDEIDEIKAHSARSGKHLQAIGKWLVPQTKHYSWLKAADIIGIGLDQVIPVPVDHNYRMDINELEKIVRGLAEEQIPVLGVVGVVGSTEEGAVDSIDKIIALRDELMKDGIYYYVHVDAAYGGYGRAIFLDEDNNFIPYEDLQDVHEEYGVFKEKKEHISREVYDAYKAIELAESVTIDPHKMGYIPYSAGGIVIQDIRMRDVISYFATYVFEKGADIPALLGAYILEGSKAGATAASVWAAHHVLPLNVAGYGKLIGASIEGSHHFYNFLNDLTFKVGDKEIEVHTLTHPDFNMVDYVFKEKGNDDLVAMNKLNHDVYDYASYVKGNIYNNEFITSHTDFAIPDYGNSPLKFVNSLGFSDEEWNRAGKVTVLRAAVMTPYMNDKEEFDVYAPKIQAALQEKLEQIYDVK